(19) United States
(12) Patent Application Publication
(10) Pub. No.: US 2010/0291045 A1
(43) Pub. Date: Nov. 18, 2010

(54) DYNAMIC VIBRATIONAL METHOD AND DEVICE FOR VOCAL FOLD TISSUE GROWTH

(75) Inventors: Xinqiao Jia, Newark, DE (US); Mingde Jia, Newark, DE (US); Amit K. Jha, Newark, DE (US); Alexandra J.E. Farran, Boothwyn, PA (US); Zhixiang Tong, Newark, DE (US)

Correspondence Address: RATNERPRESTIA, P.O. BOX 1596, WILMINGTON, DE, 19899, US

(73) Assignee: University of Delaware, Newark, DE (US)

(21) Appl. No.: 12/781,305

(22) Filed: May 17, 2010

Related U.S. Application Data
(60) Provisional application No. 61/178,717, filed on May 15, 2009.

(51) Int. Cl. A61K 35/12; C12N 13/00; C12N 5/02

(52) U.S. Cl. 424/93.7; 435/173.8; 435/325; 435/395; 435/405; 435/289.1

(57) ABSTRACT

Dynamic vibrational methods and devices for inducing differentiation of stem cells into vocal fold fibroblast-like cells or for generating vocal fold-like tissue from cultured cells. Also provided are matrices providing sustained release of growth factors, and bioreactors generating and delivering a high frequency vibration with in-plane shear stress to cultured cells.

### RELATED APPLICATIONS

This application claims priority under 35 U.S.C. §120 to U.S. Provisional Application No. 61/178,717, filed on May 15, 2009, the contents of which are incorporated by reference herein, in their entirety and for all purposes.

### STATEMENT AS TO FEDERALLY SPONSORED RESEARCH

The invention disclosed herein was made with Government support under Grant No. R01DC008965 from National Institute of Deafness and Other Communication Disorders, National Institutes of Health. Accordingly, the U.S. Government has certain rights in this invention.

### BACKGROUND OF THE INVENTION

Vocal fold, when driven into a wave-like motion by the air from the lung, produces a great variety of sounds that colors our lives. The biomechanical function of vocal fold is a direct result of its unique, laminated structure consisting of squamous epithelium, lamina propria (LP) and vocalis muscle. The lamina propria plays a critical role in the production of voice as its shape and tension determine the vibratory characteristics of the vocal folds. Under normal conditions, vocal folds can sustain up to 30% strain at frequencies of 100 to 1000 Hz. Numerous stimuli can lead to vocal fold dysfunction and damage, including excessive mechanical stress, smoke inhalation, acid reflux, allergies and inflammation, surgical accidents and cancer. Scarred vocal folds exhibit reduced sensitivity, flexibility, and diversity of the normal tissue. More severe vocal fold diseases, such as vocal fold paralysis and tumors, require drastic surgical procedures in order to restore minimum sound production.

The vocal fold fibroblasts (VFFs) are the dominant cells that are responsible for maintaining the vocal fold LP extracellular matrix (ECM) compartment in functional conditions. However, the use of primary vocal fold fibroblasts for vocal fold tissue engineering is problematic due to the lack of available healthy tissues for cell isolation. This challenge is further complicated by the fact that primary vocal fold fibroblasts cannot be readily expanded in culture beyond passage eight. Cells suitable for vocal fold tissue regeneration have not been developed.

Hyaluronic acid (HA) has been shown to play a critical role in vocal fold development and maturation. Natural HA lacks mechanical integrity and has a very limited in vivo lifetime. Chemically cross-linked HA hydrogels are bulk gels that do not recover from damage, and are not easily integrated into the host tissue. In addition, these bulk gels do not exhibit hierarchical structures that are necessary to facilitate tissue infiltration, and their degradation profile and mechanical properties cannot be adjusted without adversely affecting each other.

Growth factors (e.g., fibroblast growth factors and hepatocyte growth factors) have been found to stimulate primary VFF proliferation and ECM synthesis, and reduction of vocal fold scarring. In natural ECM, growth factors are stored as intact, latent complex through their specific binding to ECM molecules. Cell-generated tension or cell-secreted enzymes can effectively deform or degrade the latent complex and release soluble growth factors which subsequently binds their receptors causing changes in cellular functions. Growth factors have been physically encapsulated into various synthetic and natural hydrogel scaffolds to allow for diffusion-controlled passive delivery. Such strategy often results in poor temporal or spatial control of delivery due to the rapid diffusivity of growth factors through the hydrogel matrix. Growth factors have also been non-covalently immobilized in hydrogels in order to restrict their passive diffusion and permit release by other degradation mechanisms such as hydrolysis or enzymolysis. These methods, however, only allows for controlled delivery of growth factors over a short period of time varying from hours to less than a week. Covalent immobilization has been utilized to prolong the bioavailability of the growth factors to the cultured cells, but chemical transformation of growth factors inevitably compromises their biological activities.

Vocal folds are the most mechanically active tissues. During phonation, vocal fold is subjected to both normal vibration and in-plane shear stress. Titze et al. (J. Biomechanics 37(2004):1521-9; J. Biomechanics 38(2005):2406-14) have reported a bioreactor for engineering vocal fold tissues by simulating the vibrational stresses in vocal fold movement during speech. However, the Titze bioreactor does not provide both normal vibration and in-plane shear stress. The Titze bioreactor generated and transmitted vibrational strains in the 20-200 Hz frequency range to human vocal fold fibroblasts encapsulated in a three-dimensional polymer substrate via mechanical couplings of a large number of connectors and bars. Moreover, mechanical coupling may cause individual components to resonate at various frequencies, which can be detrimental to the device performance. Finally, human voice is produced when the vocal folds are blown apart into entrained vibration by the tracheal air-stream. As air is forced out of the lungs, the muscle and overlying lamina propria are driven into vibration. In other words, sound production relies on the ability of the tissue to transmit the energy between airborne acoustic waves and the organ. Variation in mechanical loading can yield diverse cellular responses and ECM remodeling.

To date, optimal treatment for vocal fold disorders has not yet been realized. There exists a need for new approaches to engineer vocal fold fibroblast-like cells and vocal fold-like tissues in an in vitro vocal fold-mimetic microenvironment for vocal fold regeneration or replacement.

### SUMMARY OF THE INVENTION

The present invention provides a method for inducing in vitro differentiation of stem cells into vocal fold fibroblast-like cells comprising (a) culturing stem cells encapsulated in a matrix, wherein the matrix comprises hyaluronic acid (HA)-based hydrogel particles (HGPs) covalently cross-linked by a water soluble polymer, and one or more growth factors bound to the HA-based HGPs, and (b) delivering a high frequency vibration with in-plane shear stress to the stem cells, wherein the matrix releases the one or more growth factors in a controlled manner effective to induce differentiation of the stem cells into vocal fold fibroblast-like cells in the presence of the vibration.

The present invention provides a method for inducing in vitro generation of a vocal fold-like tissue from cultured cells comprising (a) culturing cells encapsulated in a matrix, wherein the matrix comprises hyaluronic acid (HA)-based hydrogel particles (HGPs) covalently cross-linked by a water soluble polymer, and one or more growth factors bound to the HA-based HGPs, and (b) delivering a high frequency vibration with in-plane shear stress to the cells, wherein the matrix releases the one or more growth factors in a controlled manner effective for generating a vocal fold-like tissue from the cultured cells in the presence of the vibration.

Also provided are the vocal fold fibroblast-like cells, the vocal fold-like tissue and other biomaterials produced by the above methods. The biomaterials may comprise the matrix, the stem cells, the vocal fold fibroblast-like cells, the vocal fold-like tissue, or a combination thereof.

The present invention provides a method for regenerating vocal fold in an animal comprising transferring the vocal fold fibroblast-like cells, the vocal fold-like tissue, or the other biomaterials into vocal fold of the animal.

The present invention provides a matrix comprising (a) hyaluronic acid (HA)-based hydrogel particles (HGPs) covalently cross-linked by a water soluble polymer, and (b) one or more growth factors bound to the HA-based HGPs, wherein the matrix is capable of releasing the one or more growth factors in a controlled manner. The matrix may further comprise stem cells encapsulated in the matrix, wherein the controlled manner is effective to induce differentiation of the stem cells into specialized cells. The matrix may also further comprise cultured cells encapsulated in the matrix, wherein the controlled manner is effective to induce generation of a tissue from the cultured cells.

The present invention provides a device for generating and delivering a high frequency vibration with in-plane shear stress to cultured cells comprising (a) a cell culture chamber having the cultured cells on a membrane, (b) an acoustic pump for generating the high frequency vibration by forcing air into motion, and (c) a tube connecting the acoustic pump with the membrane for delivering the high frequency vibration with in-plane shear stress to the cultured cells via the membrane.

The present invention provides an apparatus comprising (a) a matrix comprising hyaluronic acid (HA)-based hydrogel particles (HGPs) covalently cross-linked by a water soluble polymer, and one or more growth factors bound to the HA-based HGPs, wherein the matrix is capable of releasing the one or more growth factors in a controlled manner, and (b) a device for generating and delivering a high frequency vibration with in-plane shear stress to the matrix. The device may comprise (a) a cell culture chamber having the matrix on a membrane, (b) an acoustic pump for forcing air into motion to generate the high frequency vibration, and (c) a tube connecting the acoustic pump with the membrane for delivering the high frequency vibration with in-plane shear stress to the matrix via the membrane.

The apparatus may further comprise stem cells encapsulated in the matrix and subject to the high frequency vibration with in-plane shear stress, wherein the controlled manner is effective to induce differentiation of the stem cells into vibration-sensitive specialized cells (e.g., vocal fold fibroblast-like cells, hair cell-like cells, osteoblastic cells and smooth muscle cells).

The apparatus may also further comprise cultured cells encapsulated in the matrix and subject to the high frequency vibration with in-plane shear stress, wherein the controlled manner is effective for generating a vibration-sensitive tissue (e.g., vocal fold-like tissue and inner ear tissue, muscle and bone tissues) from the cultured cells.

Also provided by the present invention are methods comprising culturing cells in the above matrix, device, or apparatus.

### BRIEF DESCRIPTION OF THE DRAWINGS

FIG. 1 shows a schematic drawing of side view of a first generation vocal fold bioreactor having two parallel vibration flasks (1: T-75 flask; 2: Connecting tube; 3: Anchoring tube; 4: Washer; 5: Cells encapsulated within the hydrogel matrix and anchored on the PDMS membrane; 6: Cell culture media; 7: Acoustic pump; 8: Power amplifier; 9: Function generator; 10: Conducting tube; 11: Acrylic stage).

FIG. 2 shows membrane normal displacement (μm) in a first generation vocal fold bioreactor as a function of applied voltage (1-7 V) and frequency (60, 110, and 300 Hz) measured by Doppler Laser Vibrometer.

FIG. 3A-3C show a second generation vocal fold bioreactor. (A) is a schematic drawing showing the bioreactor (101: Vibration chamber; 102: Bottom acrylic block; 103: Metal platform; 104: Adjustable knob; 105: T-shaped metal connecting tube; 106: Supporting column; 107: Plastic conducting tube; 108: Acoustic pump; 109: Function generator/Power amplifier. (B) is a top view of six vibration chambers assembled on one stage. (C) is a side view showing the mechanism for adjusting pre-stress in a vibration chamber.

FIG. 4 shows relative mRNA expression levels of extracellular matrix (ECM)-related genes Col I, CD44, Cox2, FN, HAS1, MMP1, TIMP1 and Elastin by MSCs cultured dynamically in a second generation vocal fold bioreactor. The cells were subjected to 1 h-on, 1 h-off vibration at 120 Hz, with a normal displacement (at the center) of 100 μm and an output voltage of 3 V after being pre-cultured for 24 h under static conditions.

### DETAILED DESCRIPTION OF THE INVENTION

Vocal fold tissue development involves unique evolution of cellular patterns in vocal folds. The vocal fold fibroblasts (VFFs) are the dominant cells that are responsible for synthesizing and degrading the extracellular matrix (ECM) as the tissue develops. At birth and shortly thereafter, there exists a relative hypercellular monolayer of cells throughout the lamina propria. By 2 months of age, there are the first signs of differentiation into a bilaminar structure of distinct cellular population densities. Between 11 months and 5 years, two distinct patterns are seen: 1) a bilaminar structure and 2) a lamina propria where there exists a third more hypocellular region immediately adjacent to the vocalis muscle. By 7 years of age, all of the specimens exhibit this transition between the middle and the deeper layers according to differential density of cell populations. A layered lamina propria structure is not present until 13 years of age and then is present throughout adolescence. As the lamina propria develops, distinct cell layers are formed. It has been postulated that the development of the vocal folds is shaped by the tasks that are required. The cellular monolayer within the LP begins to differentiate by the second month of life. Differences in the magnitudes of forces in the superficial and deeper layers of the lamina propria may induce expression of different genes in the different layers. Cell-cell communication and the evolving ECM structure and composition may also contribute to the development of vocal folds.

Along with the development of distinct cell layers is the evolution of LP ECM into the layered structure as a result of the evolving cell functions. Morphological analyses show that the vocal folds in fetuses and neonates consist of sparse and dense areas of collagen and elastic fibers, and the vocal ligament is not found. In subjects 5 years of age, a deep dense area is found in the anterior and posterior maculae flavae, and longitudinal fibers are noted between the maculae. A structure of superficial versus deep layers appears in children older than 10 years of age. The layered structure of the lamina propria is complete around 17 years of age. The development of the layered structure and the maturity of the fibers appear to reflect the complexity of phonatory function during adolescence.

The present invention is based on the discovery of a new approach for vocal fold tissue growth by an appropriate combination of cells, artificial scaffolds, biological cues and mechanical stimulation in creating an in vitro dynamic three-dimensional environment of vocal fold lamina propria for in vitro differentiation of stem cells into vocal fold fibroblast-like cells and in vitro generation of vocal fold-like tissues.

The present invention provides a method for inducing in vitro differentiation of stem cells into vocal fold fibroblast-like cells comprising (a) culturing stem cells encapsulated in a matrix, wherein the matrix comprises hyaluronic acid (HA)-based hydrogel particles (HGPs) covalently cross-linked by a water soluble polymer, and one or more growth factors bound to the HA-based HGPs, and (b) delivering a high frequency vibration with in-plane shear stress to the stem cells, wherein the matrix releases the one or more growth factors in a controlled manner effective to induce differentiation of the stem cells into vocal fold fibroblast-like cells in the presence of the vibration. Also provided are the vocal fold fibroblast-like cells and other biomaterials produced by this method. The biomaterials may comprise the matrix, the stem cells, the vocal fold fibroblast-like cells, or a combination thereof.

Stem cells are unspecialized cells that are capable of differentiating into a diverse range of specialized cells. Stem cells may be embryonic stem cells or adult stem cells. Stem cells may be pluripotent stem cells or multipotent stem cells. The stem cells may be obtained from an animal, preferably a mammal (e.g., human, monkey, pig, rabbit, ferret and mouse), more preferably a human. In some embodiments, the stem cells are multipotent mesenchymal stem cells (MSCs). MSCs are multipotent cells derived from adult bone marrow or adipose tissues.

As used herein, the term “encapsulated” means that cells are entrapped in a three-dimensional space, for example, established by the covalently interconnected polymer chains.

In some embodiments, the vibration is generated by air.

As used herein, the term “a high frequency vibration” means the vibration has a frequency in the range of phonation or audio frequencies of an animal, preferably a mammal (e.g., human, monkey, pig, rabbit, ferret and mouse), more preferably a human. Human phonation frequencies are in the range of about 20-1000 Hz. Human audio frequencies are in the range of about 15-20,000 Hz. The high frequency is in the range of about 15-20,000 Hz, preferably about 20-1000 Hz, more preferably about 60-300, most preferably about 100-300.

As used herein, the term “to induce differentiation of the stem cells” means to activate, initiate, or stimulate the stem cells to undergo differentiation, a cellular process by which cells become structurally and functionally specialized during development and adopt a specific phenotype. At least about 1%, 5%, 10%, 20%, 30%, 40%, 50%, 60%, 80%, 90%, 95% or 100%, preferably at least about 60%, more preferably at least about 80%, of the stem cells are differentiated.

As used herein, the term “vocal fold fibroblast-like cells” means cells that are differentiated from stem cells and exhibit one or more characteristics consistent with natural vocal fold fibroblasts (VFFs). Natural vocal fold fibroblasts are vocal fold fibroblasts present in a healthy animal, preferably a mammal (e.g., human, monkey, pig, rabbit, ferret and mouse), more preferably a human. Examples include primary vocal fold fibroblasts (PVFFs). The characteristics may be morphological, phenotypical or functional.

Primary VFFs (PVFFs) exhibit an elongated, spindle-shaped morphology under the phase contrast microscopy.

Phenotypical characteristics of epithelial cells, smooth muscle (or myofibroblast) cells and macrophage (CD68) are not characteristics consistent with natural VFFs. For example, the fibroblastic phenotype of PVFFs may be confirmed by a positive staining for vimentin, an intermediate filament present in fibroblastic cells and absent in epithelial cells, a negative staining for muscle specific actin (MSA) a cytoplasmic marker for myofibroblasts and smooth muscle cells and a negative staining for CD68, macrophage-specific marker.

Expression markers or expression signatures unique to natural VFFs may be used to determine whether any stem cells are differentiated into VFF-like cells. While all fibroblast cells, including VFFs, are morphologically similar, the expression pattern of ECM-related genes are different between VFFs from other fibroblast cells.

VFF unique expression markers and expression signatures can be identified to characterize PVFFs and to show the difference in genes and expression patterns between PVFFs and other related cells (e.g., MSCs, vocal fold (VF) cell lines and VFF-like cells) using conventional molecular biology techniques (e.g., genomic tools). Microarray-based gene expression analysis and Gene Ontology analysis of cells of different cell types (e.g., PVFFs, VF cell line, MSCs and neonatal foreskin fibroblasts (NFFs)) and binary comparisons are carried out to identify new genes and gene expression signatures associated with responses of these cells to vibrational forces, and to determine if these cell types undergo differentiation as may be identified by gene expression analysis.

Vocal fold fibroblast-like cells of the present invention are functional if they are capable of responding to a high frequency vibration. The responses include an expression change of an ECM-related gene (e.g., CD44, decorin, elastin (ELN), fibromodulin, fibronectin (FN), HA synthase 1 (HAS1), HA synthase 2 (HAS2), hyaluronidase 2, matrix metalloproteinase 1 (MMP1), collagen I α1, collagen I α2, collagen VI α3, tissue inhibitor of metallopeptidase 1 (TIMP1), procollagen I (ProCOLI), transforming growth factor β1, and syndican) by the cells in response to the vibration.

The present invention provides a method for inducing in vitro generation of a vocal fold-like tissue from cultured cells comprising (a) culturing cells encapsulated in a matrix, wherein the matrix comprises hyaluronic acid (HA)-based hydrogel particles (HGPs) covalently cross-linked by a water soluble polymer, and one or more growth factors bound to the HA-based HGPs, and (b) delivering a high frequency vibration with in-plane shear stress to the cells, wherein the matrix releases the one or more growth factors in a controlled manner effective for generating a vocal fold-like tissue from the cultured cells in the presence of the vibration. The cultured cells may be primary vocal fold fibroblasts (PVFFs), skin fibroblast cells or mesenchymal stem cells (MSCs).

As used herein, the term a “vocal fold-like tissue” means an aggregate of cultured cells with one or more intercellular substances organized in a way such that the aggregate exhibits a viscoelasticity in the range of that exhibited by a natural vocal fold tissue for the initiation and propogation of mucosal waves at a phonation frequency of an animal. A natural vocal fold tissue is a vocal fold tissue present in a healthy animal, preferably a mammal (e.g., human, monkey, pig, rabbit, ferret and mouse), more preferably a human.

The intercellular substances include substances present in natural extracellular matrix (ECM) of vocal fold in a healthy animal, preferably a mammal, more preferably a human. Intercellular substances include CD44, decorin, elastin (ELN), fibromodulin, fibronectin (FN), HA synthase 1 (HAS1), HA synthase 2 (HAS2), hyaluronidase 2, matrix metalloproteinase 1 (MMP1), collagen I α1, collagen I α2, collagen VI α3, tissue inhibitor of metallopeptidase 1 (TIMP1), procollagen I (ProCOLI), transforming growth factor β1, and syndican.

A vocal fold-like tissue of the present invention is functional if it is capable of responding to a high frequency vibration. The responses include vibration and sound production by the tissue in response to the vibration.

Also provided by the present invention are the vocal fold fibroblast-like cells, the vocal fold-like tissue and other biomaterials produced by the above methods.

As used herein, the term “biomaterial” means any material capable of interacting with living tissues. A biomaterial may be a tissue engineered construct comprising the matrix, the stem cells, the vocal fold fibroblast-like cells, the cultured cells, the vocal fold-like tissue, or a combination thereof. For example, a biomaterial may be a synthetic matrix in which cells (e.g., PVFFs, skin fibroblast cells and MSCs) reside.

The present invention provides a method for regenerating vocal fold in an animal comprising transferring the vocal fold fibroblast-like cells, the vocal fold-like tissue, or the other biomaterials into vocal fold of the animal. The animal is preferably a mammal (e.g., human, monkey, pig, rabbit, ferret and mouse), more preferably a human. The animal is in need of vocal fold regeneration. The animal has a vocal fold disease or disorder.

The present invention provides a matrix comprising (a) hyaluronic acid (HA)-based hydrogel particles (HGPs) covalently cross-linked by a water soluble polymer, and (b) one or more growth factors bound to the HA-based HGPs, wherein the matrix is capable of releasing the one or more growth factors in a controlled manner.

The matrix may further comprise stem cells encapsulated in the matrix, wherein the controlled manner is effective to induce differentiation of the stem cells into specialized cells. The specialized cells exhibit one or more characteristics consistent with tissue-specific cells (e.g., muscle, nerve, cartilage, adipose, salivary, vocal fold, inner ear, skin, and bone).

The matrix may also further comprise cultured cells encapsulated in the matrix, wherein the controlled manner is effective to induce generation of a tissue from the cultured cells. The tissue may be muscle, nerve, cartilage, adipose, salivary, vocal fold, inner ear, skin, and bone. Cultured cells may be cells specific for or related to the tissue to be generated, or stems cells capable of differentiation into specialized cells, which exhibit one or more characteristics consistent with tissue-specific cells. For example, the cultured cells may be dermal fibroblasts, VFFs, VFF-like cells, or MSCs.

Also provided by the present invention is a method comprising culturing cells in the matrix. The cells may be stem cells or tissue-specific cells (e.g., dermal fibroblasts, VFFs, VFF-like cells, and MSCs). The method may further comprise generating a tissue from the cultured cells.

The matrix of the present invention contains two distinct hierarchical networks (doubly cross-linked networks, DXNs): one within individual particles and another among different particles. The HA-based hydrogel particles (HGPs) are embedded in and covalently cross-linked to a secondary network. The mechanical properties (e.g., strength and stability) of DXNs are modulated by varying the particle size, surface functional group density, and intra- and interparticle cross-linking. In addition, the immobilized HA-based HGPs are utilized as drug release depots to allow for spatial and temporal presentation of biologically active compounds. The matrix offers unique structural hierarchy and mechanical properties that are suitable for in vitro soft tissue regeneration, for example vocal fold tissue generation, cartilage tissue generation, adipose tissue engineering and salivary tissue engineering.

HA-based HGPs of different sizes are prepared using conventional chemical techniques (e.g., inverse emulsion polymerization technique) to control size, improve enzymatic stability, and define surface functionality. DXNs are prepared using conventional chemical techniques, for example, by mixing water-swollen HA-based HGPs with chemically modified HA. Reactive groups are introduced to the HA-based HGPs by conventional chemical techniques for cross-linking among the particles. The reactive groups include aldehyde, hydrazide and vinyl groups. Sodium periodate oxidation may be allowed to introduce aldehyde groups to the HA-based HGPs. Carbodiimide-mediated, hydroxybenzotriazole-catalyzed coupling reaction between the carboxyl groups on HA-based HGPs and adipic acid dihydrazide (ADH) are carried out to install the hydrazide functionalities to HA-based HGPs. Vinyl groups are introduced to HA HGPs by reacting HA HGPs with glycidyl methacrylate (GMA) in aqueous media. The polymeric crosslinkers are designed to carry reactive groups similar or complementary to the surface functionality of HGPs.

Suitable water soluble polymers for cross-linking the HA-based HGPs include a HA derivative (or any biocompatible, hydrophilic polymers) carrying a hydrazide (HAADH) group, a HA (or any biocompatible, hydrophilic polymers) derivative carrying an aldehyde (HAALD), a HA (or any biocompatible, hydrophilic polymers) modified with glycidyl methacrylate (GMA), or HA (or any biocompatible, hydrophilic polymers) carrying free thiol.

HA particles are readily crosslinked using HAADH, giving rise to macroscopic gels with a simple chemical composition. The enzymatic stability of the macrogel is dependent on the crosslinks between particles rather than crosslinks within HGP since the crosslinker HAADH is more prone to enzymatic degradation. The introduction of the secondary crosslinking that exhibits controlled degradation profile is advantageous. The degradability of the secondary crosslinking network is manipulated by varying the relative concentration of HAADH and PEG-dihydrazide. Various DXNs are incubated under physiologically relevant conditions in the presence or absence of hyarluronidase, and hydrogel degradation is monitored using the carbazole assay.

The hydrogel microstructures in the matrix are characterized by conventional techniques (e.g., microscopy and neutron scattering techniques). The size and size distribution of the hydrogel particles are characterized by Coulter Counter and dynamic light scattering while the surface charge is characterized by measuring the zeta potential of the particle suspension in PBS using a Zeta Pals. The preparation and characterization of exemplary HA-based HGPs and HA-based matrices are described in detail in Jha et al., 2009, Macromolecules 42(2): 537-46; Jha et al., 2009, Biomaterials 30:6964-75; and Farran et al., 2010, Tissue Eng. 16(4):1247-61; the contents of each of which are hereby incorporated in their entirety.

The average diameter of the HA-based HGPs is about 20-500,000 nm, preferably about 900-10,000 nm, more preferably about 500-900 nm.

The average mesh size of the HA-based HGPs is about 1-100 nm, preferably about 1-20 nm, more preferably about 5.5-7.0 nm. The average mesh size is determined by various methods, including protein uptake experiment, solute exclusion, Differentiation Scanning Calorimetry (DSC), Nuclear Magnetic Resonance (NMR) and Scanning Electron Microscrope (SEM).

The HA-based HGPs have a narrow particle size distribution. Particle size distribution may be characterized by polydispersity index (PDI) (mean/median particle size). PDI can vary from about 1.0 to 4.0, preferably about 1.0-2.1, more preferably about 1.0-1.5.

The aldehyde carrying particles may be allowed to react with cascade blue hydrazide to calculate the aldehyde content by comparing the solution fluorescence before and after the reaction using a spectrafluorometer. The matrix in various embodiments of the invention may have an aldehyde content of about 5-70%, preferably about 40-60%, more preferably about 10-20%, by mole.

To quantify the hydrazide content in HGPs, the particles may be allowed to react with fluorescamine, which becomes fluorescent upon reaction with amines. The matrix has a hydrazide content of about 10-50%, preferably about 25-40%, more preferably about 30-38% by mole.

The viscoelasticity of the matrix may be selected to be similar to that of a natural tissue. The natural tissue may be muscle, nerve, cartilage, adipose, salivary, vocal fold, inner ear, skin, and bone, preferably vocal fold. It may be quantified at various frequencies. For example, the elastic modulus of the matrix may be about 10-10000 Pa, preferably about 200-2000 Pa, at human phonation frequencies.

By controlling the degree of crosslinking, robust, yet soft and pliable, hydrogel networks are obtained. Combining two crosslinking mechanisms by covalently crosslinking these hydrogels very lightly stabilizes the particles without unnecessarily increasing the modulus of the hydrogel, and provides opportunities for erosion of the hydrogels upon formation of vocal fold-like tissue in vitro. In doing so, the degradation properties are decoupled from mechanical responses of HA that make up the network. The mesh size of the primary, intraparticle, network (i.e., HA-based HGPs) is about 1-100 nm, preferably about 1-20 nm, more preferably about 6-7 nm. The average pore size for the secondary, interparticle, network may be as large as several hundred nanometers, for example, about 20-500 nm, preferably about 20-300 nm, more preferably about 50-150 nm. This structure provides unique capabilities, including entrapping and releasing growth factors from different microscopic locations within the hydrogel matrix.

The matrix may further comprise other macromolecules, including collagen, gelatin and fibronectin. Collagen and HA are two of the most abundant ECM molecules in vocal fold LP, and can recapitulate the native microenvironment of vocal fold LP when strategically combined. The incorporation of covalently cross-linkable HA derivatives into self-assembled collagen fibers not only enhances the integrity of composite gels but also allows for fine-tuning of their structure and mechanics. Elastic, macroscopic gels are formed in less than 30 seconds upon mixing of 1wt % aqueous solutions of HACHO and HAADH, stable hydrazone bonds are formed, and water is released as the only by-product. The crosslinking chemistry, combined with the biocompatible nature of HA constituents, makes these derivatives attractive candidates for in situ cell encapsulation. HA/collagen hydrogels are synthesized by combining chemically modified, covalently cross-linkable HA derivatives with self-assembling collagen monomers. Collagen monomer is combined with HA derivatives carrying aldehyde groups (aldehyde-carrying HA; HAALD) and hydrazide groups (HA modified with adipic acid dihydrazide (ADH); HAADH) to form chemically defined composite hydrogels.

The matrix of the present invention provides spatio/temporal presentation of growth factors or growth factor morphogens bound to HA-based HGPs, which act as growth factor depots, and release growth factors in a controlled manner. As used herein, the term “controlled manner” means that growth factors are released from HA-based HGPs over a prolonged period of time. The matrix provides a cumulative release (e.g., no more than about 100%, 99%, 95%, 90%, 80%, 70%, 60% and 50%) of growth factors for a prolonged period of time (e.g., at least 1, 4, 7, 10, 15, 20, 25, 30 or 35 days). The cumulative release of growth factors is preferably no more than about 100%, more preferably no more than about 99%, even more preferably no more than about 95%, for at least 7 days, more preferably at least 10 days, even more preferably at least 15 days. For example, the growth factors may be released in a steady state at about 3.8% growth factor per day over a period of at least 15 days. The preparation and characterization of exemplary matrices capable of releasing growth factors in a controlled manner are described in detail in Jha et al., 2009, Macromolecules 42(2): 537-46; and Jha et al., 2009, Biomaterials 30:6964-75; the contents of each of which are hereby incorporated in their entirety.

Suitable growth factors include basic fibroblast growth factor 2 (bFGF or FGF2), hepatocyte growth factor (HGF), bone morphogenetic proertin-2 (BMP-2) and connective tissue growth factor (CTGF), transforming growth factor β1, preferably FGF2 and HGF.

Growth factors bind to HA-based HGPs directly or via linking molecules. Linking molecules include extracellular matrix (ECM) molecules, peptide analogues and RNA aptamers. ECM molecules include heparan sulfate proteoglycans (HSPGs), heparin, and unique domains thereof (e.g., Perlecan Domain I (PlnDI)). These linking molecules are used as ligand reservoirs for storage and release of growth factors, protecting these proteins from inactivation by proteolytic digestion and potentiating their biological functions. The linking molecules may be covalently immobilized to HA-based HGPs for non-covalent sequestration of growth factors via cross-linking polymers. Suitable cross-linking polymers include poly(ethylene glycol) (PEG) linkers, and poly(L-glycine).

For example, growth factors may be immobilized in the doubly crosslinked networks via specific binding with perlecan domain I (PlnDI) proteoglycan that has been previously anchored to the hydrogel particles. Immobilization of growth factors within the matrix enhances their biological activities and availability. Both TGF and FGF bind to PlnDI with a high affinity and specificity and their mitogenic activity, biological stability and availability are modulated by PlnDI binding. Heparan sulfate present in PlnDI serves as co-factors to promote binding of FGF to high affinity receptors, enhancing its activity. HGF also binds PlnDI to act as a tumor suppressor, morphogen, and angiogenic factor.

The PlnDI-conjugated HGPs are used as the growth factor stores, allowing growth factors (e.g., FGF-2 and HGF) to form latent complexes through specific binding to PlnDI. To maximize its growth factor binding capacity, PlnDI can be covalently conjugated to HGPs through its core protein via a flexible PEG linker. Both the core protein and the HS side chains are available for effective growth factor binding. The binding and release of growth factors from the matrix are monitored in vitro via immunochemical assays (ELISA) and radiolabelling assays in order to determine the optimal loading condition. The biological activities of the released growth factors (HGF-2 and bFGF) are assessed indirectly by evaluating their effects on MSC proliferation, differentiation and ECM production. Cell number is determined using a Cell Proliferation Kit with fluorescence plate reading and DNA picogreen assay.

The development of HA hydrogels for cell growth and tissue remodeling has been impeded by poor cell attachment. Protein deposition and cell attachment are thermodynamically unfavorable because of the hydrophilic and polyanionic properties of HA. The matrix of the present invention further comprises a bioactive compound. Bioactive compounds include antifibrotic drugs, cell adhesive modules and cytokines.

Antifibrotic drugs are drugs used to prevent the formation of fibrous tissues. Examples include mitomycin C and dexamethasone.

Cell adhesive factors may be immobilized to the HA-based HGPs using conventional techniques to enhance cell adhesion and to increase the effectiveness of immobilized growth factors. Cell adhesive factors may be peptides, including fibronectin-derived GRGDSP domains, fibronectin-mimetic peptide (Ac—KSSPHSRN(SG)₅RGDSPCONH₂), N-terminal aminooxy peptide (NH₂—O—CO-GSSPHSRN(SG)₅RGDSP—CONH₂), C-terminal peptide aldehyde (Ac-GSSPHSRN(SG)₅RGDSP—NH—C(R,H)CHO) and cysteine-containing peptide (Ac—CGGGGGGGRGDSPGGGGGYGGE-CONH₂). The as-synthesized peptide (or crude product) is subjected to HPLC and ESI-MS analysis for the chemical identities and purity. These peptides react readily with aldehyde groups via oxime ligation, leading to the formation of a hydrazone bond that is relatively stable under physical conditions. The FN-mimetic peptide (Ac—KSSPHSRN(SG)₅RGDSPCONH₂) may be conjugated to HGPs via the lysine amine using the aldehyde groups on particle surfaces by reductive amination. The N-terminal aminooxy peptide (NH₂—O—CO-GSSPHSRN(SG)5RGDSP—CONH₂) and C-terminal peptide aldehyde (Ac-GSSPHSRN(SG)₅RGDSP—NH—C(R,H)CHO) react readily with aldehyde groups via oxime ligation, leading to the formation of a hydrazone bond that is relatively stable under physical conditions. Reduction of the hydrazone bond by NaBH₃CN stabilizes the linkage. The presence of an adhesive peptide is quantified by hydrolyzing the peptide from the particles. The peptide concentration is analyzed by amino acid analysis (AAA), ninhydrin test, UV absorbance and NMR analysis.

In order for the scaffold to effectively transmit forces into the encapsulated cells, the cells need to adhere to a matrix with an optimal affinity. Cells cultured on soft hydrogels (E about 1 kPa) show diffuse and dynamic adhesion complex, whereas those cultured on stiff substrates (Young's modulus (E) 30-100 kPa) have stable focal adhesion. MSCs are defined as plastic-adherent cells from the bone marrow, and require adhesive substrate for survival and phenotype maintenance. On the other hand, if the adhesion is too strong, they won't be able to respond to the external forces dynamically. Thus, the adhesive strength between MSCs and peptide decorated HGPs is optimally tuned by varying the peptide density and their proximity on the particle surfaces. The adhesive strength of the matrix may be about 10-60 Pa, preferably about 40-55 Pa.

The adhesive strength is measured by culturing cells on 2D hydrogel surface and in 3D hydrogel matrices in a serum-free media. MSCs seeded on the gel surface (10⁴ cells/cm²) or in the hydrogel scaffolds (10⁶ cells/mL) are evaluated by confocal microscope after the nuclei and cytoskeletal F-actin are fluorescently labeled with DRAQ5™ and Alexa Fluor 488 Phalloidin, respectively. For example, cells cultured on 2D hydrogel surfaces are imaged using an inverted phase contrast microscope and projected cell areas are measured. Alternatively, the adhesive strength between MSCs and ligand-modified HGPs are characterized by exposing confluent cells on a glass slide to fluorescently labeled HGP in a flow chamber. The surface associated fluorescence is assessed as a function of time and position with fluorescence microscopy.

The DXNs in the matrix provides 3D culture for cells encapsulated in the matrix. Matrices with enhanced biological functions are formed by direct mixing of constituent building blocks, for example, PlnDI-conjugated HGPs that are loaded with a selected growth factor, RGD decorated HGPs, as-synthesized HGPs and soluble crosslinkers. The hydrazone bond is formed rapidly in isotonic solutions, with water being the only by-product. The concentration of each component may be systematically adjusted in order to obtain matrices with the optimal stiffness and desired biological functions.

The biocompatibility of HA-based HGPs and water soluble crosslinkers is evaluated by culturing cells in the presence of the particles or the crosslinkers under standard culturing condition using Dulbecco's modified Eagle's medium (DMEM) that contains 10% fetal bovine serum (FBS) and 100 Units/mL penicillin and 100 μg/mL streptomycin. The final particle or crosslinker concentration is adjusted to vary from 0.002 to 2.0 mg/ml. Cytotoxicity is qualitatively investigated using a live/dead cell viability assay that provides two-color fluorescence assessment based on the simultaneous determination of live and dead cells with two probes that measure recognized parameters of cell viability-intracellular esterase activity and plasma membrane integrity. Alternatively, the relative toxicity is quantitatively assessed with a commercially available MTT viability assay, in which purple formazan produced by active mitochondria is quantified spectroscopically. Cell proliferation rates are determined by prolonged incubation time using the same assays. Cell morphology is closely monitored via microscopy. The HA concentration in the supernatant culture media is examined by enzyme-linked immunosorbant assay (ELISA) over a period of time, for example, two weeks, with the culture media being changed every other day without disturbing the hydrogel particles. The presence of HGF in the culture media released from the engineered hydrogel particles leads to elevated HA production over prolonged period of time, and results are compared to control groups where HGF is directly administered into the culture media.

To evaluate cell cytotoxicity in 3D culture, trypsinized cells are mixed with the water soluble crosslinker with defined concentration of reactive hydrazide functionality. HA-based HGPs are separately dispersed in cell culture media until fully swollen. Using a two-barrel syringe, the cell suspension is directly mixed with the HGP suspension, and the mixture is subsequently passaged within the syringe for several times to ensure homogenous mixing and efficient gelation. Alternatively, cells can be entrapped in the gels by a photocrosslinking process. The relative amount of HGPs and the crosslinker may be systematically varied in order to obtain a cell/gel construct that is not only homogeneous but also, importantly, has viscoelastic properties similar to that of the natural vocal fold LP. The degree of homogeneity is qualitatively assessed via LSCM for each construct by constructing Z-stack LSCM images of cell stained with cell tracker green. If the gelation is not fast enough, most of the cells sediment at the bottom. Cell viability is confirmed immediately after the encapsulation using trypan blue exclusion or live/dead staining. The gel is then be cultured in cell culture insert under static condition for 28 days in cell culture media. Cell attachment is confirmed by cytoskeleton F-actin staining. A spinal-shaped, spread morphology indicates cell attachment. Vinculin staining may be used to reveal focal adhesion associated proteins that function as a linker between actin filament and integrin. Hydrogels containing scrambled peptide sequence may be used as the controls. Cell morphologies are closely monitored using phase contrast microscope and confocal microscope.

The present invention also provides a device or bioreactor for generating and delivering a high frequency vibration with in-plane shear stress to cultured cells. The device may be constructed by conventional methods in various ways to generate well-defined vibrational and shear stresses that closely mimic the mechanical forces exposed to the vocal folds. The device comprises (a) a cell culture chamber having the cultured cells on a membrane, (b) an acoustic pump for generating the high frequency vibration by forcing air into motion, and (c) a tube connecting the acoustic pump with the membrane for delivering the high frequency vibration with in-plane shear stress to the cultured cells via the membrane.

As used herein, the term “membrane” means a thin layer. The membrane is capable of delivering, transferring or transmitting vibration. The membrane may be flexible and elastomeric. For example, the membrane may be composed of poly(dimethyl siloxane) (PDMS). The membrane may be coated with collagen I, fibronectin, poly(L-lysine) and/or laminin to enhance cell adhesion.

The membrane may have a tensile strength in the range of about 1.6-9.0, preferably about 5.5 MPa; a Young's modulus in the range of about 230-1500 KPa, preferably about 1100 KPa; an elongation to break in the range of about 120-430%, preferably about 250%; and a tear strength in the range of about 4.9-37.7, preferably about 13.1 kN/m.

The membrane is positioned to transfer the vibration to the cells with in-plane shear stress. In-plane shear stress is the stress along the surface of the membrane. As the membrane is being vibrated in the normal direction, it is being stretched along the membrane direction. Stress is force per unit area. Strain is percent deformation.

The membrane exhibits pre-stress (strain) before the vibration is delivered to the membrane through the tube because the membrane can be stretched prior to the initiation of the vibrational stimulation.

The cell culture chamber is capable of providing a condition suitable for culturing cells. The cell culture chamber may be a tissue culture flask or an acrylic chamber.

The acoustic pump is a means capable of forcing air into motion to generate a high frequency vibration. The acoustic pump may include a speaker enclosed in a wooden chamber. The tube may be made of any material capable of delivering a high frequency vibration. The vibration may have a frequency in the range of phonation or audio frequencies of an animal, preferably a mammal (e.g., human, monkey, pig, rabbit, ferret and mouse), more preferably a human.

The device further comprises a means for adjusting the pre-stress, the frequency and amplitude of the vibration. Pre-stress can be adjusted by the rotating knob or adjustable knob. The frequency can be adjusted by the function generator and the amplitude can be tuned by the power amplifier (by changing the output voltage).

Also provided by the present invention is a method comprising culturing cells in the device. The cultured cells may be vibration-sensitive cells, for example, cells from vocal fold, skin, inner ear, and bone, muscle, or stem cells capable of differentiation into vibration-sensitive cells.

As used herein, the term “vibration-sensitive cells” means cells that respond to the vibration. The response may include changes in cell morphology, cell phenotypes, expression markers, or expression signatures in response to the vibration. For example, the cultured cells may be stem cells, MSCs, VFFs, hair cells, skin fibroblasts, smooth muscle cells or osteoblasts. The cells may be cultured in a matrix on the membrane in the cell culture chamber.

The method further comprises generating a vibration-sensitive tissue in vitro from the cultured cells.

As used herein, the term a “vibration-sensitive tissue” means an aggregate of cultured cells with one or more intercellular substances organized in a way such that the aggregate exhibits a viscoelasticity in the range that exhibited by a natural tissue in response to the vibration. The vibration-sensitive tissue may be a tissue exhibiting one or more characteristics consistent with a natural vibration-sensitive tissue, for example, vocal fold, inner ear, muscle or bone.

The bioreactor of the present invention may be used to characterize vibration-sensitive tissues or cells, to identify genes related to disorders or diseases associated with these tissues or cells, and to screen for potential therapeutical candidates for treating the disorders or diseases. Exemplary disorders or diseases include loss of voice, loss of hearing, and loss of normal functions of skin, muscles, bone, blood vessels or nervous systems.

The present invention provides an apparatus comprising (a) a matrix comprising hyaluronic acid (HA)-based hydrogel particles (HGPs) covalently cross-linked by a water soluble polymer, and one or more growth factors bound to the HA-based HGPs, wherein the matrix is capable of releasing the one or more growth factors in a controlled manner, and (b) a device for generating and delivering a high frequency vibration with in-plane shear stress to the matrix. The device may comprise (a) a cell culture chamber having the matrix on a membrane, (b) an acoustic pump for forcing air into motion to generate the high frequency vibration, and (c) a tube connecting the acoustic pump with the membrane for delivering the high frequency vibration with in-plane shear stress to the matrix via the membrane.

The apparatus may further comprise stem cells encapsulated in the matrix and subject to the high frequency vibration with in-plane shear stress, wherein the controlled manner is effective to induce differentiation of the stem cells into vibration-sensitive specialized cells.

The vibration-sensitive specialized cells may be cells exhibiting one or more characteristics consistent with natural vibration-sensitive cells, for example, vocal fold fibroblasts, hair cells, osteoblasts and smooth muscle cells.

The apparatus may also further comprise cultured cells encapsulated in the matrix and subject to the high frequency vibration with in-plane shear stress, wherein the controlled manner is effective to induce generation of a vibration-sensitive tissue from the cultured cells.

The vibration-sensitive tissue may be a tissue exhibiting one or more characteristics consistent with a natural vibration-sensitive tissue, for example, vocal fold, inner ear, muscle or bone.

Also provided by the present invention are methods for culturing cells in the apparatus. The cultured cells may be vibration-sensitive cells or stem cells capable of differentiation into vibration-sensitive cells.

The practice of the present invention will employ, unless otherwise indicated, conventional techniques of organic chemistry, polymer chemistry, molecular biology, microbiology, virology, recombinant DNA technology, immunology, mechanical devices, and electrical devices, which are within the skill of the art.

As used in this specification and the claims, the singular forms “a,” “an,” and “the” include plural references unless the content clearly dictates otherwise.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate to perform the disclosed methods.

### Example 1

### Hyaluronic Acid Doubly Cross-Linked Networks

In this experiment, a new class of hyaluronic acid (HA)-based hydrogel materials was created with HA hydrogel particles (HGPs) embedded in and covalently cross-linked to a secondary network. The HA-based HGPs were synthesized and used for creating macroscopic gels, which contain two distinct hierarchical networks (doubly cross-linked networks, DXNs): one within individual particles and another among different particles. Bulk gels (BGs) formed by direct mixing of HA derivatives with mutually reactive groups were included for comparison. The materials and methods used in this experiment are described in Jha et al., 2009, Macromolecules 42(2): 537-46, the contents of which are hereby incorporated in their entirety

DXNs with measurable modulus were formed in less than 2 h upon mixing the newly synthesized particles. In the absence of oxHGP, HAADH solution (2 wt %) is simply a viscous fluid that does not have any mechanical integrity. The addition of oxHGP locks in the network structure, giving rise to a stable hydrogel that does not dissolve in an aqueous buffer. The majority of HAADH was effectively interconnected to the particles in the hydrogel matrix as confirmed by the relatively low sol fraction (14.8±3.1%) for DXNs. Contrarily, the bulk gels exhibited a sol fraction as high as 54.0±3.3%, in agreement with previous results on fast gelling hydrogel systems. Again, the bulk gel was formed within minutes of mixing, whereas the formation of stable DXNs takes 1-2 h. The longer times required for gelation resulted in more efficient crosslinking in DXNs. In other words, DXNs contained a very small amount of physically entangled HAADH that did not contribute to the overall stability of the gels. The optical microscopy image of the as-synthesized DXNs showed a large amount of interconnected hydrogel particles throughout the matrix.

The microstructure of DXNs was characterized locally by cryoSEM and globally by SANS/USANS. Bulk gels (BGs) prepared using soluble HAADH and HAALD were included as the controls. For cryoSEM study, samples were prepared under high pressure and rapid freezing conditions, which effectively led to in situ vitrification of water within the hydrogel samples. Subsequent sublimation of the vitrified water revealed the internal structures of the hydrogel that could be visualized by cryoSEM. It was evident that both the bulk gel and the DXN were composed of porous, interconnected network structures. The microstructure of DXNs was distinctly different from that of the bulk due to the presence of embedded oxHGPs. While dry HGPs were perfectly spherical particles with relatively smooth surfaces, the particles in DXNs exhibited undulated surfaces. Unlike traditional composite hydrogel where the particles were simply physically trapped in the hydrogel matrix, the DXNs had distinctive hierarchical structures, whereby the secondary network originated from the particle surfaces covalently. A close inspection of the cryoSEM image for DXNs indicated a diffuse interphase between individual oxHGPs and the matrix, which proved that the secondary network originated from the particle surface. As a result, the secondary network could exert mechanical constraints on the hydrogel particles, leading to the deformation of HGP through the covalent hydrazone linkage between the secondary network and the HGP surface. It was also evident that the HGPs were much less porous than the secondary network where they were embedded.

SANS data for BGs as well as the DXNs were collected for the scattering intensity (I(q)) vs. the scattering vector (q), where q=(4n/λ)×sin(θ/2) (λ: wavelength of incident neutrons; θ: scattering angle). A quantitative estimation of the hydrogel network morphology (100's nm) could be made by a comparison of the low q scattering exponent. A scattering exponent in the low q regime could be interpreted to be representative of the network density. The bulk gel had an exponent of 2.3, indicative of a mass fractal. Mass fractal scattering occurs when I(q)˜S(q)˜q^{-Dm}, where Dₘ is the mass fractal dimension and has a value of 1-3. This value increased to 2.9 for DXN, suggestive of an increase in the compactness/density of the network resulting from the densely cross-linked particles, absent earlier. Analyses on other hydrogel and biophysical systems produced similar changes in the fractal nature of self-assembled networks with concentration.

The combined SANS and USANS data for the 0.5 wt % HGPs covered four orders of magnitude in length scales from a few Å to 10's of microns. The plateau in the intensity seen at the low q regime is typical of scattering from spherical objects and has often been seen in nano/micro particles, and in micellular nanostructures from surfactants and block copolymers. A Guinier analysis of the data in the low q saturation regime gave a particle radius of 452 nm. This global analysis was in excellent agreement with local dimensions extracted from SEM data and those from DLS, which estimated the particle diameter to be around 900 nm and confirmed the relative monodispersity of the particles.

Due to the presence of large amounts of aldehyde groups, oxHGPs effectively served as the cross-linking and branching points in the network. Individual particles were interconnected by HAADH and continued to grow and randomly branched out in the same fashion as the polymer chains in traditional bulk gels. Thus, the micron-sized, interparticle spacing seen in the cryoSEM image originated from the HAADH-threaded particles.

The mechanical properties of HA DXNs were characterized. The viscoelastic properties of DXNs as well as BGs were measured at low frequencies (<10 Hz) using a commercial rheometer in oscillatory shear mode, and at high frequencies (>10 Hz) using a home-built torsional wave apparatus. For the rheology measurements, standard mineral oil was applied around the geometry to prevent samples from drying out during the measurement. All measurements were carried out at 37° C. using a peltier plate. Similarly, in torsional wave experiments, the hydrogel samples, sandwiched between two vertically-aligned plates, were enclosed in an environmental chamber with a controlled temperature (34-37° C.) and humidity (>94% RH). Both measurements were performed within the linear viscoelastic region: the applied strain was 0.2% for oscillatory rheological tests, and the imposed rotational angle was 0.2° in the torsional wave experiments.

At frequencies of 0.1-10 Hz, the elastic and loss modulus for both types of hydrogels were time and frequency-independent, confirming the covalent nature of the matrices. This stability indicated that the permanent chemical cross-links were not destroyed by the increasing frequency (or time) at a constant strain (0.2%), These solid-like, elastomeric materials exhibited G′ values approximately two orders of magnitude higher than the corresponding G″. The average storage moduli were 930 Pa and 390 Pa for DXNs and BGs, respectively, while the average loss moduli were 24 Pa and 4 Pa for DXNs and BGs, respectively. The damping ratio (tan(δ)) for DXNs was an order of magnitude higher than that for BGs. Collectively, at frequencies below 10 Hz, DXNs were stiffer and more energy dissipative as compared to BGs. The effect of particles size and size distribution on the overall viscoelasticity of DXNs can be readily observed by comparing the current DXN systems with those reported previously. DXNs generated using less-defined particles had an average G′ of 510 Pa and G″ of 8 Pa; both values were lower than the gels prepared using smaller and more mono-disperse particles.

It has been demonstrated that the viscoelasticity of hydrogel samples at phonation frequency range could be determined accurately and reliably using a torsional wave apparatus. This technique relies on analyzing the amplification factor around the resonance frequency of the hydrogel samples, having the geometry of circular disks, using an optical lever technique combined with linear viscoelastic wave analysis. The amplification factors for HA-based DXNs were frequency-dependent. Model prediction was based on the average values from three consecutive measurements on the same sample. The hydrogel samples was loaded between rigid plates that were aligned vertically. The linear viscoelastic wave analysis provided a good fit to the observed frequency-dependence of the amplification factor at the resonance frequency. The calculation indicated that an increase in the frequency at which resonance occurred could be affected by decreasing the thickness of the sample. In other words, by changing sample geometry, one could deduce the viscoelastic properties at a wide range of frequencies.

The viscoelastic properties of DXNs and BGs were frequency-dependent. Based on the results obtained from oscillatory rheology and torsional wave analyses, both BGs and DXNs became stiffer when the frequency of the applied mechanical perturbation increased from <10 Hz to ≧50 Hz. The average elastic modulus for BGs increased from 390 Pa at frequencies below 10 HZ to 495 Pa at 50-80 Hz, reaching 754 Pa at resonance frequencies in the range of 100-160 Hz. Similarly, the average elastic modulus for DXNs increased from 930 Pa at low frequencies to 1144 Pa at resonance frequencies of 50-70 Hz. The tan(δ) values remain essentially the same across the frequency range investigated for BGs and DXNs. Their values were relatively small—near the lower limit of the measurements—for both the low and high frequency experiments.

### Example 2

### Hyaluronic Acid Hydrogel Particles With Enhanced Biological Functions

In this experiment, a biomimetic growth factor delivery system was developed for spatial and temporal presentation of a biologically active growth factor, bone morphogenetic protein 2 (BMP-2), to effectively stimulate chondrogenic differentiation of cultured mesenchymal stem cells (MSCs). In particular, sustained release of biologically-active BMP-2 was accomplished using PlnDI-conjugated HA-based HGPs. Chondrogenic differentiation of MSCs was demonstrated using micromass cultures of C3H10T1/2 cells in the presence of BMP-2-loaded, PlnDI-conjugated HA-based HGPs. Materials and methods used in the experiment are described in Jha et al., 2009, Biomaterials 30:6964-75, the contents of which are hereby incorporated by their entirety.

Compared to HGP without PlnDI, HGP-P₁ exhibited significantly (p<0.05) higher BMP-2 binding capacity and distinctly different BMP-2 release kinetics. Heparitinase treatment increased the amount of BMP-2 released from HGP-P₁, confirming the HS-dependent BMP-2 binding. While BMP-2 was released from HGPs with a distinct burst release followed by a minimal cumulative release, its release from HGP-P₁ exhibited a minimal burst release followed by linear release kinetics over 15 days. The bioactivity of the hydrogel particles was evaluated using micromass culture of multipotent MSCs, and the chondrogenic differentiation was assessed by production of glycosaminoglycan, aggrecan and collagen type II. BMP-2 loaded HGP-P₁ stimulated more robust cartilage specific ECM production as compared to BMP-2 loaded HGP, due to the ability of HGP-P₁ to potentiate BMP-2 and modulate its release with a near zero-order release kinetics. The HA-based, PlnDI-conjugated HGP provides an attractive growth factor delivery system for tissue repair and regeneration.

HA-based HGPs were synthesized by in situ crosslinking of HAALD and HAADH within the inverse emulsion droplets. The resulting HGPs were spherical in shape and had an average particle size of 10 μm. HA-based HGPs were chemically and enzymatically more stable than their macroscopic counterparts (bulk gels made with the same chemical composition). No degradation was observed after the particles were soaked in buffers at pH of 4 or in the presence of hyaluronidase (100 U/mL at pH 7.4) for one month, whereas the bulk gel completely disintegrated overnight under the same conditions.

The average FITC-dex retention as a function of the molecular weight followed a bell-shape distribution, and the lowest retention was observed for FITC-dex-4 (13.4±6.8%) and FITC-dex-2000 (25.5±10.8%). The highest retention was observed for probes of intermediate sizes. The smallest probe, FITC-dex-4, diffuses readily in and out of the particles, resulting in an overall low retention. On the other hand, the largest probe was excluded from the majority of the pores in the particles, and only approximately 25% of pores had comparable sizes to FITC-dex-2000, thus being retained. When the average mesh size of the hydrogel particles became comparable to the size of the probe molecules, a high percentage (84-86%) of the probe molecules that were taken up by the particle remained trapped in the particles during the second equilibrium process. Because the Stokes radius for FITC-dex-40 and FITC-dex-250 was 4.5 nm and 10.5 nm, respectively, it was estimated that the average mesh size of the hydrogel particles was in the range of 5-10 nm.

A predictive structural analysis of PlnDI core protein including the conserved SEA domain showed that although the core protein folded into a defined secondary structure with a hydrophobic core, the charged lysine residues (Lys 85, 97, 104 and 117 in the mature secreted form of Pln) were well exposed on the surface, thus readily accessible for covalent conjugation. The fifth lysine residue (Lys 164) was not shown in the 3D structure. The as-synthesized HGPs first were treated with glycine to convert the free aldhyde groups on the particles to carboxylic acid prior to the conjugation reaction. The glycine-treated particles were allowed to react with large excess of PEGdiALD, converting the residue hydrazide groups into the aldehyde groups that were extended from the surface of the nanopores by approximately 77 ethylene glycol repeats. A positive CB staining confirmed the presence of aldehyde groups in HGP and HGP-PEG. The negative CB staining for glycine-treated HGPs indicated the effectiveness of free glycine to block the reactive aldehyde groups. HGP-PEG subsequently was reacted with PlnDI under acidic conditions at a solution pH slightly below the pI value of PlnDI (4.26). Lysine amines were employed to establish the covalent linkage between the core protein and the PEG-modified HGPs by reductive amination.

The PlnDI-conjugated HA-based HGPs (HGP-P₁), along with the as-synthesized HGPs, were subjected to particle size analysis using a Coulter Counter Multisizer. Under isotonic conditions, the majority of the hydrogel particles appeared to be 5-6 μm in diameter, smaller than they appeared to be microscopically. Upon PEG modification and PlnDI conjugation, the average particle size increased to around 8 μm, as indicated by the shift in the size distribution profile for HGP-P₁ relative to that for HGP. Such a distinct and consistent shift does not necessarily mean a net particle size increase of ˜2 μm upon PlnDI conjugation. Rather, it implies the altered ability of the electrolyte to pass freely through the hydrogel particles, which can be attributed to the presence of covalently anchored PlnDI heparan sulfate “brushes” within the nanopores of HGP-P₁. Particles also were analyzed by Alcian blue staining to identify their GAG content. HGP-P₁ showed positive Alcian blue staining performed at pH 1.0, whereas HGP remained negative. The robust blue stain seen for HGP-P₁ confirmed the presence of GAG side chains on PlnDI, and hence the presence of PlnDI in HGPs. The immobilized PlnDI retained its ability to bind BMP-2. In this experiment, BMP-2 loaded particles were incubated with HRP-conjugated anti-human BMP-2. Upon addition of the colorimetric reagents, HGP-P₁-B₂ gave rise to deep blue staining, whereas HGP-B₂ remained colorless under the microscope although the particle suspension exhibited a faint blue hue. The difference in the degree of BMP-2 association between HGP and HGP-P₁ further confirms the presence of covalently immobilized PlnDI in HGP-P₁.

BMP-2 ELISA analysis showed that for every milligram of hydrogel particles, 168±3 ng BMP-2 was associated with HGP-P₁ in contrast to only 89±10 ng for HGP, suggesting a higher binding capacity of HGP-P₁. This calculation agrees well with the qualitative BMP-2 binding results by Alcian blue staining. In order to assess the effect of enzymatic treatment on BMP-2 release, various hydrogel articles with loaded BMP-2 were subjected to a 2-h heparitinase treatment and the BMP-2 concentration in the supernatant was analyzed using BMP-2 ELISA. Hydrogel particles without the loaded BMP-2 were included as the controls. The heparitinase treatment did not change the overall amount of BMP-2 released from HGP. However, BMP-2 release from HGP-P₁ was significantly (p<0.05) modulated by the enzyme activity, with heparitinase treatment more than doubling the amount of BMP-2 released into the supernatant. In the absence of heparitinase, both HGP and HGP-P₁ released similar amounts of BMP-2 after 2-h incubation. Taken together, these findings indicate that PlnDI was stably immobilized to HA-based HGPs and its presence greatly promoted HS-dependent BMP-2 binding to HA HGP-P₁.

In vitro BMP-2 release from HGP and HGP-P₁ was evaluated by incubating BMP-loaded hydrogel particles in a physiological buffer for up to 15 days. After 3 days, 30±7 ng of BMP-2 was retained by one milligram of HGP, corresponding to a cumulative release of 66±3%. After 15 days of incubation, one milligram HGP retained only 4±5 ng BMP-2, with a total of 95±5% of initially bound BMP-2 being released. In contrast, a sustained release with a reduced initial burst was observed for HGP-P₁. After 3 days, 129±4 ng of BMP-2 was retained by a total of one milligram HGP-P₁, indicating a cumulative release of 23.6±0.3%. After 15 days of incubation, one milligram of HGP-P₁ retained 51±4 ng of BMP-2, releasing 69.5±0.5% of BMP-2 initially bound. The experiments were terminated before 100% release was achieved. Examination of the release curves suggests that BMP-loaded HGP exhibited a release profile with two distinct slopes (43% per day vs 2.2% per day), indicating a variable release rate and the presence of an initial burst release. On the other hand, PlnDI-conjugated HGPs maintained a steady state of BMP-2 over the entire course of the experiments with a cumulative release of 3.8%/day over 15 days of incubation. Collectively, these results confirm that PlnDI, when conjugated to HGPs, permits a close to zero-order release kinetics. The long-term zero-order release of the growth factor, BMP-2, would allow tissues at the injection site to be exposed to the growth factor at a constant concentration for a long period, allowing sufficient time for generation of functional new tissues.

C3H10T1/2 cells were plated at a high density in direct contact with various particles, including HGP, HGP-B₂, HGP-P₁, and HGP-P₁-B₂, to assess their chondrogenic potential. Micromass cultures displayed intense Alcian blue staining when incubated in the presence of HGP-P₁-B₂ and HGP-B₂, indicating GAG accumulation in both cases. HGP-P₁-B₂ treated micromass exhibited more robust blue staining and the cells were confined in a circle with a distinct boundary. On the other hand, HGP-B₂-treated micromass displayed a dark, but diffuse, staining with a ill-defined cell boundary. Interestingly, micromass cultured with carriers alone (HGP-P₁ and HGP) was stained weakly positive by Alcian blue. The HGP treated micromass was bigger in diameter and more diffuse in overall appearance than that treated with HGP-P₁. Cells cultured in control media exhibited the least Alcian blue staining.

Chondrogenic differentiation was further evaluated by immunohistological analyses for aggrecan and collagen type II, two well accepted markers for mature hyaline cartilage tissues. In both analyses, the cell nuclear DNA was stained green while the cell-secreted aggrecan and collagen type II were stained red. Non-specific binding of the secondary antibody alone was negative. No significant aggrecan production was detected for the vehicle-treated and the untreated micromass culture. Aggrecan and collagen II accumulation was detected in the micromass cultured in the presence of BMP-2-loaded particles, both HGP-P₁ and HGP. However, HGP-P₁-B₂ stimulated the cells to produce larger amount of aggrecan and collagen type II than the HGP-B₂, as evidenced by the more striking red stain for HGP-P₁-B₂-treated micromass cultures. Syto 13 staining revealed that cells in all micromass cultures exhibited a spherical-shaped morphology, indicative of the absence of cell differentiation into spread osteoblastic or fibroblastic phenotype.

### Example 3

### Hyaluronic Acid-Based Hydrogel Matrices With Enhanced Biological Functions

In this experiment, biomimetic hydrogel matrices were developed not only to exhibit structural hierarchy and mechanical integrity, but also to present biological cues in a controlled fashion. To this end, photocrosslinkable, hyaluronic acid (HA)-based hydrogel particles (HGPs) were synthesized via an inverse emulsion crosslinking process followed by chemical modification with glycidyl methacrylate (GMA). HA modified with GMA (HA-GMA) was employed as the soluble macromer. Macroscopic hydrogels containing covalently integrated hydrogel particles (HA-c-HGP) were prepared by radical polymerization of HA-GMA in the presence of crosslinkable HGPs. The covalent linkages between the hydrogel particles and the secondary HA matrix resulted in the formation of a diffuse, fibrilar interface around the particles. Compared to the traditional bulk gels synthesized by photocrosslinking of HA-GMA, these hydrogels exhibited a reduced sol fraction and a lower equilibrium swelling ratio. When tested under uniaxial compression, the HA-c-HGP gels were more pliable than the HA-p-HGP gels and fractured at higher strain than the HA-GMA gels. Primary bovine chondrocytes were photoencapsulated in the HA matrices with minimal cell damage. The 3D microenvironment created by HA-GMA and HA-based HGPs not only maintained the chondrocyte phenotype but also fostered the production of cartilage specific extracellular matrix. To further improve the biological activities of the HA-c-HGP gels, bone morphogenetic protein 2 (BMP-2) was loaded into the immobilized HGPs. BMP-2 was released from the HA-c-HGP gels in a controlled manner with reduced initial burst over prolonged periods of time. The HA-c-HGP gels are promising candidates for use as bioactive matrices for cartilage tissue engineering.

### Materials and Methods

Hyaluronic acid (HA, sodium salt, ˜500 kDa) was obtained from Genzyme Corporation (Cambridge, Mass.). Dioctyl sulfosuccinate sodium salt (Aerosol OT, AOT, 98%), 1-heptanol (1-HP), 2,2,4-trimethylpentane(isooctane), bovine testicular hyaluronidase (HAase, 30,000 U/mg), divinyl sulfone (DVS), (N,N-dimethylamino)pyridine (DMAP), tetrabutylammonium bromide (TBAB), 2,2-dimethoxy-2-phenylacetophenone (DMPA), 1-vinyl-2-pyrrolidinone (NVP), Alcian blue 8GX and glycidyl methacrylate (GMA) were purchased from Aldrich (Milwaukee, Wis.). Acetone, concentrated sulfuric acid, sodium hydroxide, carbazole and ethanol were obtained from Thermo Fisher Scientific (Waltham. Mass.). Recombinant human BMP-2 (rhBMP-2) and BMP-2 Quantikine ELISA kit were obtained from R&D Systems (Minneapolis, Minn.). Paraformaldehyde (16% in H₂O) was obtained from electron microscopy sciences (Hatfield, Pa.). Dulbecco's minimum essential medium (DMEM), fetal bovine serum (FBS), penicillin, streptomycin, and glutamine were purchased from Invitrogen (Carlsbad, Calif., USA). Propidium iodide and SYTO 13 were purchased from Genway Biotech, Inc (San Diego, Calif.). All reagents were used as received.

HA was chemically modified with GMA following previously reported procedures (Jia et al., Macromolecules, 2004, 37, 3239-3248). To an aqueous solution of HA (15 mg/mL, 50 mL) was added DMAP (6.4 molar excess relative to HA repeats), GMA (20 molar excess) and TBAB (2.7 molar excess). The reaction was allowed to proceed under nitrogen at room temperature in the dark for 48 h. Upon addition of NaCl, the reaction mixture was added drop-wise to a large access of acetone. The white, fluffy precipitate was collected by filtration. The precipitation process was repeated twice and the final precipitate was re-dissolved in 300 mL H₂O and dialyzed (MWCO 10,000) against 0.1 N NaCl for 24 h and DI H₂O for another 24 h. The purified product was freeze dried and stored at 4° C. in the dark prior to use.

To synthesize HA-based HGPs, HA was allowed to react with DVS in inverse emulsion droplets of aqueous NaOH dispersed in isooctane and stabilized by AOT and 1-HP. After 1 h reaction, a large amount of acetone was added to the reaction mixture and HGPs were obtained by extensive washing and repeated centrifugation at 12,000 rpm, followed by drying under vacuum at room temperature. To render the resulting HGPs photocrosslinkable, the particles were dispersed in DI H₂O at a concentration of 10 mg/mL. To this suspension was added GMA (40 molar excess relative to the HA repeats), along with DMAP (40 mol %) and TBAB (80 mol %). The reaction was allowed to proceed at room temperature in a nitrogen atmosphere for 96 h in the dark. The methacrylated HGPs (HGP-GMA) were washed with NaCl (5 wt %) solution, water, ethanol and acetone three times. The vacuum dried particles were stored at 4° C. in the dark prior to use.

HA-c-HGP gels were prepared using HA-GMA and HGP-GMA as the macromer and crosslinker, respectively. Four mg of dry HGP-GMA were dispersed in DI H₂O and the swollen particles were collected after the centrifugation at 12,000 rpm for 10 min. The particle pellet was re-dispersed in HA-GMA (0.5 mL, 2 wt %) containing 1.5 μL of the initiator solution (30% DMPA in NVP). The solution was exposed to a long wavelength UV lamp (Model 100AP, Blak-Ray) for 10 min for complete gelation. The HA-p-HGP gels were prepared similarly using HGP instead of HGP-GMA. Traditional bulk gels (HA-GMA) were synthesized in the absence of hydrogel particles. Briefly, MAGMA (1 mL, 2 wt % in DI H₂O) was mixed with the initiator solution (3.0 μL) and the solution was subjected to UV irradiation for 10 min.

¹H NMR spectra were recorded in D₂O on a Bruker AV400 NMR spectrometer under standard quantitative conditions and were analyzed with Bruker Topspin software. All chemical shift values were calibrated using the solvent peak (from proton impurities, 4.8 ppm for D₂O).

The average size and size distribution of hydrogel particles (dispersed in DI H₂O) were analyzed by dynamic light scattering (DLS) using Zetasizer Nano ZS (Malvern Instruments, UK). Three separate injections were analyzed and the graph reported was a representative trace. Scanning electron microscopy (SEM) images of HGP-GMA were obtained using a JSM 7400F SEM (operating voltage: 3 kV, current: 10 μA). Particles suspended in acetone were deposited on aluminum stubs, and the acetone was evaporated at room temperature.

Cryogenic SEM (CryoSEM) was applied to visualize the native hydrogel microstructure. Samples were frozen rapidly (≧10 000 deg/s) under high pressure (≧2000 bar) and then freeze fractured in liquid nitrogen. After water was sublimed, the specimens were coated with gold-palladium (10 mA current) at −130° C., and subsequently viewed in a Hitachi S-4700 FESEM (Tokyo, Japan) at 1 kV (emission current: 30 μA) with a working distance of approximately 3-6 mm.

To measure the hydrogel sol fraction, the as-synthesized gels were allowed to swell in PBS (pH of 7.4). After 24 h of incubation at 37° C., PBS was aspirated, and the gel disks were soaked in DI H₂O for another 24 h at 37° C. Hydrogels were thoroughly washed with DI water to remove the excess salt. Samples were subsequently dried by passing through graded ethanol solutions. The sol fraction was calculated from the ratio of the final dry weight and the initial solid mass used in the gel preparation. To measure the equilibrium swelling ratio, the as-synthesized gels were dried by passing through degraded ethanol solutions, and the solvent was evaporated under vacuum. After the dry weight was recorded, the gel discs were immersed in PBS at 37° C. overnight and the wet weight was measured. The swelling ratio was defined as the ratio of the final wet weight to the initial dry weight. The results reported were an average from five repeated measurements.

Mechanical analysis was conducted on a dynamic mechanical analyzer (RSA III instrument, TA Instruments, New Castle, Del.) using a parallel plate geometry that measures the deformation as a function of normal stress. The gel disks (height: 4.0 mm and diameter: 6.3 mm) were prepared in the cell culture insert and compression tests were performed immediately upon gelation. All samples were compressed at a rate of 20%/min until fracture. Compression tests were performed in triplicate for all samples. The modulus was calculated using the initial linear portion of the stress-strain curve.

The stability of the hydrogels and hydrogel particles was evaluated in the presence of HAase. Individual hydrogel disks (˜1.3 mg dry weight) were separately immersed in a HAase solution (1 mL, 5 U/ml) in PBS at pH 7.4 at 37° C. HGPs (3 mg) were incubated at 37° C. in a HAase solution (1 mL, 100 U/mL) in sodium acetate buffer at pH of 4.0. The supernatant was aspirated off every other day and was stored at −20° C. until further analysis. The degradation medium was replenished with freshly prepared enzyme solution. The amount of HA degraded was quantified by carbazole assay following reported procedures. All analyses were carried out in triplicate and the cumulative degradation (as a % of total) was calculated by dividing the amount of uronic acid released up to a chosen measurement time with the initial dry weight the gels.

Bovine articular cartilage was dissected from carpal joints of legs of ˜1-year-old bovines and was shipped from Indiana overnight on dry ice. Immediately upon arrival, the explants were minced and subsequently digested in a Pronase solution (1 mg/mL in DMEM with 10% FBS) for 30 min at 37° C. The explants were subsequently washed with PBS twice and incubated in a Collagenase P solution (1 mg/mL, in DMEM with 10% FBS) at 37° C. overnight under gentle agitation at 50 rpm. The digested solution was passed through a 40 or 70 μm filter, and centrifuged for 8-10 min at approximately 200 g. The supernatant then was aspirated off, and the pellet was washed twice with PBS. The cell pellet was subsequently suspended in DMEM/F-12 medium containing 1% PS, 10% FBS, 50 μg/mL sodium pyruvate and 50 μg/mL ascorbic acid. The chondrocyte viability was determined using trypan blue exclusion and dead cells (blue) were counted manually using a hemacytometer. Primary chondrocytes were plated at a confluency of 50-60% and incubated at 37° C. in a humidified atmosphere with 5% CO₂ with the complete media for 24 h and serum starved for another 24 h before the experiment.

For cell culture studies, the dialyzed HA-GMA solution was sterile filtered (0.22 μm) before being lyophilized. Hydrogel particles were exposed to a germicidal lamp in a laminar flow hood for 30 min, followed by soaking in ethanol/H₂O (70%) overnight. Chondrocytes were dispersed in the precursor solutions that contained HA-GMA, HGPs and photoinitiator at a final concentration of 4×10⁶ cells/mL. The suspension was transferred to a cell culture insert that was placed in a 24 well plate, and was exposed to the UV irradiation for 10 min. The cell-laden hydrogels were incubated at 37° C. in a humidified atmosphere with 5% CO₂ with 400 μL complete media. Medium was refreshed every 3 days. After 7 days of culture, the cell/gel constructs were stained with propidium iodide (1:2000 in DPBS) and SYTO 13 (1:1000 in DPBS). Images were acquired using a Zeiss Axiovert confocal microscope (Axiovert LSM 510).

The production of sulfated glycosaminoglycan (sGAG) was confirmed calorimetrically using an Alcian blue staining. After 9 days of culture, the cell/gel constructs were washed with PBS and fixed in 4% (v/v) paraformaldehyde for 4 h. The samples subsequently were stained with Alcian blue (0.5 wt % in 3% (v/v) glacial acid) at pH 1.0. After overnight incubation at ambient temperature, the constructs were thoroughly rinsed with distilled water and were photographed with a digital camera attached to a Nikon microscope (Coolpix 990, Nikon, Kanagawa, Japan). Cell-free hydrogels were subjected to the same staining protocol as the controls.

The BMP-2 loading buffer was prepared from PBS, with the addition of 0.1% BSA, 1% Penicillin-Streptomycin (PS) and 0.1 mM phenylmethylsulfonyl fluoride (PMSF). One mg of hydrogel particles were dispersed in 200 μL of loading buffer containing 200 ng rhBMP-2. The suspension was left overnight at 4° C. The BMP-2 loaded hydrogel particles were collected by centrifugation at 12,000 rpm for 10 min and the supernatant was removed for BMP-2 quantification using BMP-2 ELISA kit following the manufacture's instruction. The amount of BMP-2 loaded into the hydrogel particles was determined by subtracting the amount of BMP-2 remaining in the supernatant from that initially added. BMP-2 loaded particles thus prepared were immediately used for hydrogel formation following procedures described above. For BMP-2 release study, hydrogel disks or particles were incubated in the release media (CMRL 1066, with 1% PS, 400 μL) at 37° C. At predetermined time points, the supernatant was collected and an equal amount of release media was added. The amount of BMP-2 in the release medium was measured using the BMP-2 ELISA kit. All measurements were carried out in triplicate.

All quantitative measurements were performed on 3 replicates. All values are expressed as means±standard deviations (SD). Statistical significance was determined using a two-tailed student t-test. A p value of less than 0.05 was considered to be statistically different.

### Results

The goal of the current investigation was to engineer HA-based hydrogels that are hierarchically structured, mechanically robust and biologically active, suitable for use as 3D matrices for in vitro culture of differentiated primary chondrocytes. The hydrogel systems synergistically combine the unique attributes of microgels and traditional bulk gels by photocrosslinking HA hydrogel particles with a soluble, HA macromomer (HA-GMA). Photocrosslinking is advantageous over other chemical approaches because it permits rapid gelation under physiological temperature and pH with accurate spatial controls. Direct photoencapsulation of cells in a synthetic hydrogel creates a construct with defined nanoscale topography and tunable viscoelasticity that promote control of cell-matrix interaction, cell differentiation, cell migration and neotissue formation. Photocrosslinking is an efficient method for encapsulation and growth of various types of cells including chondrocytes and embryonic stem cells.

Synthesis and Characterization of Photocrosslinkable HA-based HGPs. Soluble HA macromer (HA-GMA) was synthesized successfully following established procedure (Jia et al., Macromolecules, 2004, 37, 3239-3248). An 11% methacrylation was determined by comparing the relative integrations of the vinyl protons (˜5.6 and ˜6.1 ppm) and HA's methyl proton (NAc—CH₃, ˜1.8 ppm). Separately, HA-based HGPs were synthesized by covalent crosslinking of HA with DVS within the inverse emulsion droplets of water/isooctane that were stabilized by AOT and 1-heptanol. The crosslinking occurs between the primary OH groups of HA and the vinyl groups in DVS under basic conditions (U.S. Pat. No. 4,636,524). The resulting HA-based HGPs were spherical in shape and exhibited smooth surfaces and an average diameter of ˜1 μm. HA-based HGPs were subsequently methacrylated (HGP-GMA) by reacting with a large excess of GMA in DI H₂O in the presence of tertiary amine and a phase transfer agent. Both trans-esterification reaction between the residual, primary hydroxyls in HGPs and the ester group of GMA, and the carboxylate-initiated ring-opening conjugation with GMA exist under the reaction conditions employed. The presence of hydrated, nano-scale pores in HA-based HGPs, combined with the small molecular dimension of GMA and the presence of phase transfer agent, suggest that the chemical modification occurred throughout the bulk of the particles, rather than being limited to the particle surfaces.

To confirm the presence of the methacrylate groups in HGP-GMA, particles were solublized in acidic medium prior to ¹H NMR analysis. HGPs and GMA treated under the same conditions were included as the controls. The ¹H NMR spectrum for the hydrolyzed HGPs presented the characteristic signals of the degraded HA products, possibly consisting of oligosaccharides of various molecular weights. No addition reaction occurred between HCl and the unsaturated double bond in GMA under the degradation conditions since the vinyl protons are clearly present (˜5.7 and ˜6.2 ppm) and the integration ratio between the vinyl protons and the methyl protons (CH₃, 2 ppm) was close to 2/3. Simple mixing of GMA with HA-based HGPs in the absence of the catalyst and the phase transfer agent did not lead to the conjugation of the methacrylate group as evidenced by the absence of the vinyl protons in the degraded particles. In the presence of DMPA and TBAB, HGPs were successfully methacrylated after 96 h of reaction. The characteristic vinyl protons, although shifted to a higher field, were clearly visible after HGP-GMA was degraded. The shift of the vinyl protons as compared to GMA was a consequence of the alteration of the chemical environment upon degradation and chemical conjugation. A simple gelation experiment was conducted to further confirm the crosslinkability of HA HGP-GMA. Specifically, when acrylamide was radically polymerized in the absence of HGP-GMA, a polyacrylamide solution was obtained. When HGP-GMA was introduced in the monomer solution, a viscoelastic gel was formed. Collectively, the results confirmed that HGPs were properly methacrylated.

Typical scanning electron micrographs of HGP-GMA confirmed the presence of well-defined spherical particles with relatively narrow size distribution. Quantitative particle size analysis by DLS revealed an average diameter of 1.00±0.08 μm for HGP-GMA. The DLS traces for HGP and HGP-GMA essentially overlapped, indicating that the chemical modification did not alter the size and structure of the hydrogel particles. Particle degradation is not likely to occur during the modification process. The crosslinkable HGPs can be consistently synthesized and the dry particles can be readily dispersed in water.

Hydrogel Microstructure. With HA-GMA and HGP-GMA in hand, the formation of 3D macroscopic hydrogels via a radical polymerization was investigated. In the presence of UV irradiation, DMPA fragmented into radicals that propagated along HA-GMA and HGP-GMA via a chain mechanism. The resulting hydrogels contained HA chains, HGPs and kinetic chains of poly(methacrylic acid). Stable hydrogel disks with defined shapes were obtained after 10 min of UV irradiation. While the HA-GMA gels were optically clear, the inclusion of micron sized hydrogel particles in the HA-GMA gave rise to semi-opaque gels, with HGPs homogeneously distributed throughout the secondary gel matrix. Prolonged incubation of both types of gels in DI H₂O did not alter their physical appearance, indicating the absence of particle diffusion and hydrogel hydrolysis.

Although HA-p-HGP and HA-c-HGP-gels appear similar to the naked eye, their structures differed at microscopic levels, CryoSEM was employed in place of traditional SEM so as to preserve the innate hydrogel microstructures without creating artifacts during sample preparation. Typical CryoSEM images for HA-GMA, HA-p-HGP and HA-c-HGP gels showed that the HA-GMA gels were composed of homogeneously distributed pores of submicron size range. Both HA-p-HGP and HA-c-HGP gels contained densely crosslinked, nanoporous HGPs surrounded by a loosely interconnected, secondary matrix that exhibited similar morphology as the HA-GMA matrix. When HGPs were physically entrapped in HA-GMA matrix, a depletion zone around the hydrogel particles was created even though the HGPs and the secondary network were of the same chemical make-up. In the case of HA-c-HGP gels, the secondary network was connected to the particle covalently as evidenced by the fibrilar structure bridging the particle surface and the secondary matrix. A diffuse interphase between individual particles and the secondary matrix was created. The highly reactive radicals, once generated photochemically, immediately reacted with the nearest and the most accessible π-bond to form the propagating radicals. It is highly unlikely for the HA chains carrying the reactive radicals to diffuse deep into the HGPs during the gelation process because of the large molecular dimensions of branched or partially crosslinked HA-GMA, the small mesh size of HGPs, the short half life of radicals and the rapid reaction kinetics. Collectively, the results confirmed that the radical species along the HA-GMA chains were capable of propagating through the methacrylate groups anchored on the particle surface, establishing covalent linkages between the particles and the surrounding, secondary matrix.

Hydrogel Swelling and Sol Fraction. The equilibrium water uptake by the hydrogels was measured after overnight immersion in PBS at 37° C. Hydrogel sol fraction was evaluated by comparing the dry mass of the hydrogels before and after 24-h equilibration in PBS, during which the physically entangled HA chains that are not immobilized to the network (sol fraction) diffused out of the gels. The HA-GMA gels exhibited an average swelling ratio of 62±3.6 and a sol fraction of 34±13%. Physical inclusion of HGPs in the HA-GMA matrix led to a reduction in both swelling ratio (43±4) and sol fraction (25±10%). Hydrogels containing covalently integrated HGP-GMA exhibited the lowest water uptake (38±2). Close to 90±5.8% of macromers were effectively incorporated in the final HA-c-HGP gel networks.

Hydrogel Degradation and Mechanics. In natural ECM, hyaluronidase (HAase) is responsible for HA turnover, which can affect cell migration, differentiation, and matrix catabolism. HAase catalyzes the random scission of 1,4-linkages between 2-acetamido-2-deoxy-b-D-glucose and D-glucose residues in native HA. The optimal pH for HAase is 4.5-6. The enzymatic stability of HGPs and various hydrogels were monitored by carbazole assay and the cumulative degradation profiles were plotted as the overall percentage of uronic acid detected as a function of degradation time. At low HAase concentration under physiological pH, minimal degradation was detected for HGPs. To accelerate the degradation, HGPs were suspended in 100 U/mL in a sodium acetate buffer at pH 4.0. The percent degradation as a function of incubation time showed an initial linear loss of HA at a rate of ˜9% per day up to 6 days. Thereafter, the degradation rate leveled off. Approximately 25 wt % of HGPs was left after 15 days of degradation.

When exposed to 5 U/mL HAase at pH 7.4, the bulk gels progressively became smaller and more fragile. The gel disks lost their coherence and were fragmented after 6 days of incubation. Approximately 73.0±12.5% degradation was detected at day 4 and complete degradation was observed at day 10. Incorporation of HGPs in the HA-GMA matrix, both physically and covalently, resulted in composite matrices with improved enzymatic stability. Both types of gels degraded at a slower rate than the HA-GMA gels, and the uronic acid produced at day 4 accounted for approximately 55.4±4.6% and 46.0±2.5% of the initial weight HA, respectively. Unlike the bulk gels, particle-containing hydrogels maintained their macroscopic geometry and structural integrity throughout the course of the degradation study. The opaque gel disks remained present on day 15, although their size and thickness were decreased.

The ability of HAase to degrade the HA-based hydrogels suggests that the chemical modification and covalent crosslinking did not significantly alter the biological identity of HA. A swelling ratio of 62 for the HA-GMA gels, as compared to 5 for the HGPs, clearly indicates a less crosslinked network for the former. The presence of residual surfactant molecules on the particle surface may limit the ability of HAase to access the particles for surface erosion. It is speculated that the hydrogels degrade predominately via surface erosion mechanism due to the diffusion restriction of HAase into the interior of the gel. The addition of densely crosslinked HGPs in the bulk HA-GMA matrix naturally results in an enhanced enzymatic stability. The covalent coupling between HGP-GMA and the HA-GMA further enhances the enzymatic stability of the resulting matrices. Degradation characteristic of hydrogel matrices plays an essential role in modulating cellular functions. The motivation in combining the two cross-linking mechanisms stems from an interest in only very lightly covalently crosslinking these hydrogels to stabilize the particles without unnecessarily increasing the modulus of the hydrogel, and to provide opportunities for erosion of the hydrogels upon regeneration of native tissues in vivo.

Mechanical properties of HA-GMA, HA-p-HGP and HA-c-HGP gels were examined. In particular, representative stress versus strain curves were obtained for these HA-based hydrogels. Linear viscoelastic regions were observed at strains <20%. The up-turn, commonly observed in hydrogel materials at high strain (>30%), could be attributed to the material densification and/or strain hardening. The compressive modulus, calculated by taking the slope of the stress-strain curve in the linear region, for HA-GMA, HA-p-HGP and HA-c-HGP gels was 15.7±1.8 kPa, 22.7±1.2 kPa and 18.5±1.2 kPa, respectively. The compressive modulus for HA-p-HGP was significantly different from that for HA-GMA, p≦0.05; *: The compressive modulus for HA-p-HGP was significantly different from that for HA-c-HGP, p≦0.05. The addition of densely crosslinked hydrogels particles to HA-GMA matrix led to a moderate increase in the compressive modulus and fracture strain.

Unlike the traditional composites where the fillers are stiff, inorganic nanoparticles, the inclusion of small amount (21% of the total mass) of hydrogel particles into a soft hydrogel matrix did not lead to a dramatic change in matrix stiffness. Hydrogels containing physically trapped HGPs were statistically stiffer (p≦0.05) than those containing covalently integrated particles. When the HA-p-HGP gels were compressed, the mechanical force applied to the gels could not be directly transmitted to the entrapped particles due to the absence of particle-matrix coupling and the presence of the depletion zone around the particles. On the other hand, the presence of the covalent linkages between the individual particles and their surrounding matrix in HA-c-HGP gels permitted the external forces to be directly transmitted to the particles, resulting in particle deformation without compromising the overall matrix strength. Force-induced particle deformation was observed in a HA-based, doubly crosslinked networks prepared by direct mixing of aldehyde presenting HGPs and a soluble HA derivative carrying hydrazide groups.

3D Chondrocyte Culture. The potential of HA-c-HGP hydrogels was evaluated for in situ encapsulation and 3D culture of primary bovine chondrocytes. Live/dead assay after 7 days of culture detected minimal numbers of dead cells, indicating the biocompatible nature of the encapsulation process as well as the resulting matrices. Furthermore, cells retained chondrocytic spherical morphology in 3D, whereas those cultured on 2D TCPS adopted a spread-out, spindle shaped morphology, suggesting differentiation. Chondrocytes were dispersed uniformly throughout the gels without severe aggregation as encountered in other macroporous HA gels. The production of sulfated glycosaminoglycan (sGAG) was analyzed by Alcian blue staining. Alcian blue is known to selectively stain sGAG rather than HA at low pH. The control HA gels without the cells showed a minimal background staining. The Alcian blue staining for HA-p-HGP and HA-c-HGP gels revealed the presence of HGPs homogeneously distributed in the secondary matrix, in agreement with visual inspection. The cell/gel constructs exhibited intense and robust blue staining, indicative of sGAG accumulation and neocartilage formation. Chondrocytes encapsulated in HA-GMA gels were clearly visible. Because the embedded cells appeared similar microscopically to the hydrogel particles, both in size and shape, they could not be easily identified in the composite matrices by Alcian blue staining. All three types of hydrogel investigated exhibited similar levels of blue staining, irrespective of their composition and microstructure.

Freshly isolated bovine articular chondrocytes underwent de-differentiation to a fibroblast-like phenotype when cultured in monolayer. When encapsulated in the 3D microenvironment created by HA and HGPs, chondrocytes were able to re-differentiate and express their original phenotype. In the absence of soluble factors or biophysical stimulations, primary chondrocytes residing in the HA matrix produced remarkable amounts of sGAG within a short culture time. Therefore, the HA matrices described here not only provided mechanical support for the cells but also actively modulated their in vitro behaviors through cell-matrix interaction.

Growth Factor Release. BMP-2 releasing matrices were prepared by photocrosslinking of HA-GMA in the presence of BMP-2-loaded HGPs (77.5±4.0 ng per mg HGPs). The presence of loaded BMP-2 did not interfere with the gelation process. Stable gel discs with the same compression properties were obtained after 10 min UV irradiation. Protein exclusion experiments using stable, globular proteins with molecular dimensions ranging from 3.0 to 10.2 nm revealed that HA-based HGPs had an average pore size of 5.5-7.0 nm. With an apparent molecular weight of 26 kDa and a pI value of 8.21, BMP-2 was readily driven into the interior of HGPs via ion exchange.

In vitro BMP-2 release from HA-p-HGP and HA-c-HGP gels was evaluated by incubating the hydrogel disks in a physiological buffer for up to 15 days in the absence of any enzyme. BMP-2 loaded HGPs were included as the controls. The BMP-2 release kinetics strongly depended on the hydrogel microstructures. The release curves for HGPs and the HA-p-HGP gels were characterized with an initial burst from day 0 to day 3, followed by a release at a much slower rate over 10 days. Physical encapsulation of BMP-2 loaded particles in a secondary matrix introduced additional pathways for the growth factor molecules to diffuse through, hence a shift of the overall release curve to a lower cumulative release at any given time points. After 3 days, a cumulative release of 68.9±5.4% and 46.1±0.9% was observed for HGPs and P-gels, respectively. After 13 days of incubation, a total of 84.9±9.0% and 52.3±0.7% of initially bound BMP-2 was released from HGP and HA-p-HGP gels, respectively. The slower release observed after the initial burst was due to the association of HA with BMP-2 at pH 7.4, presumably due to the ionic bonds between the negatively charged HA and positively charged BMP as well as multiple hydrogen-bonding interactions between HA and BMP-2.

Contrarily, when BMP-2 loaded HGPs were covalently integrated in a secondary, HA-based matrix, a sustained release with a significantly reduced initial burst was observed. After 3 days of incubation, only 10.3±6.4% BMP-2 initially loaded BMP-2 was released. After 15 days of incubation, 55.7±10.2% of BMP-2 still remained bound to the matrix. The experiments were terminated before 100% release was achieved. Overall, the HA-c-HGP gels maintained a steady state of BMP-2 over the entire course of the experiments with a cumulative release of 3.0%/day.

### Example 4

### Primary Vocal Fold Fibroblasts in Three-Dimensional Hyaluronic Acid-Based Hydrogel Matrices

In this study, the applicability of chemically defined collagen/HA composite hydrogels was evaluated for use as conducive, 3D matrices for in vitro static culture of vocal fold fibroblasts. Two types of collagen and hyaluronic acid (HA)-based hydrogels were synthesized. They were compositionally similar, but structurally variable and mechanically different. Type A hydrogels were composed of mature collagen fibers reinforced by oxidized HA, while Type B hydrogels contained immature collagen fibrils interpenetrated in an amorphous, covalently cross-linked HA matrix. PVFFs encapsulated in either matrix adopted a fibroblastic morphology and expressed genes related to important ECM proteins. DNA analysis indicated a linear growth profile for cells encapsulated in Type B gels from day 0 to day 21, in contrast to an initial dormant, non-proliferative period from day 0 to day 3 experienced by cells in Type A gels. At the end of the culture, similar DNA content was detected in both types of constructs. A reduction in collagen content was observed for both types of constructs after 28 days of culture, with Type A constructs generally retaining higher amounts of collagen than Type B constructs. The HA content in the constructs decreased steadily throughout the culture, with Type A constructs consistently exhibiting less HA than Type B constructs. Using the torsional wave analysis, it was found that the elastic moduli for Type A constructs decreased sharply during the first week of culture, followed by two weeks of matrix stabilization without significant changes in matrix stiffness. Conversely, the elastic modulus for Type B constructs increased moderately over time. Materials and methods used in this study are described in Farran et al., 2010, Tissue Eng. 16(4):1247-61; the contents of which are hereby incorporated in their entirety.

Composite matrices based on self-assembled collagen and HA derivatives containing mutually reactive functional groups (HACHO and HAADH) offer the opportunity to fine-tune the matrix properties. Two types of composite hydrogels were assessed in this study. Type A gels were formed by mixing HACHO with self-assembling collagen monomers, while Type B gels were prepared by addition of HAADH to the premixed HACHO/collagen solution. Hydrogels based on collagen and LMW virgin HA, as well as collagen only, were included as the controls. CryoSEM images showed that all four types of hydrogels were composed of interdigitated fibrillar structures with pores in the nanometer scale. The microstructure of collagen/HA gels and collagen/HACHO gels closely resemble that of the collagen gels, consisting mainly of entangled fibrils of ˜50-100 nm in diameter and several microns long. A close inspection of the cryoSEM image for Type B gels reveals subtle differences in its microstructure from other composite hydrogels investigated. While fibrillar entities are clearly present, they are less defined and exhibit a smaller overall fiber diameter. The distinct interstitial phase resembles the microstructure of the amorphous gels formed by direct mixing of NACHO and HAADH. While the fibers in other gels follow straight, strut-like pathways, the fibrils in Type B gels are readily bent and appeared more flexible. Individual fibrils seem to be interconnected by an amorphous matrix with smaller pore sizes. Small Angle Neutron Scattering (SANS) analysis (Supporting Information), on the other hand, did not reveal significant structural differences for all matrices investigated.

Freshly isolated PVFFs exhibited an elongated, spindle-shaped morphology as revealed by the phase contrast microscopy. The fibroblastic phenotype of PVFFs prior to the encapsulation was confirmed by a positive staining for vimentin, an intermediate filament present in fibroblastic cells and absent in epithelial cells, and a negative staining for muscle specific actin (MSA), a cytoplasmic marker for myofibroblasts and smooth muscle cells. In situ encapsulation of PVFFs in collagen/HA gels was achieved by mixing PVFFs with soluble HA derivatives and collagen followed by 45-min incubation at 37° C. The encapsulation process was highly compatible since the majority of cells remained viable immediately after cell encapsulation as assessed by the live/dead assay. PVFFs cultured in both types of composite matrices exhibit the same staining patterns for vimentin (positive) and MSA (negative) as those freshly isolated from the tissue. No gel compaction could be visually detected for both types of constructs until the end of culture when a minor reduction of construct diameter was observed.

Cells maintained their ability to proliferate within the 3D hydrogels as indicated by the increase in cell number with the days of culture. By day 28, both types of gels had been densely populated by the proliferating PVFFs. Occasionally, isolated dead cells were detected in the gels. F-actin staining was applied to cells encapsulated in 3D hydrogels in order to assess cell morphology and cytoskeleton organization. Several hours after encapsulation (day 0), actin filaments remained clustered around the nuclei, giving rise to a rounded morphology. The majority of the cells remained rounded after one day of culture and an additional day of culture was necessary for most cells to attach and spread within the matrices. By day 3, PVFFs developed distinctly long and organized cytoskeleton processes suggesting their involvement in cell attachment. The proliferating PVFFs maintained their elongated morphology throughout the matrix during the course of culture. Comparable cell morphology and cell distribution patterns were observed in both types of hydrogels. PVFFs cultured in 3D composite hydrogels seem to be more elongated than those cultured on 2D plastic surfaces.

Cell proliferation was quantified by analyzing the DNA content per construct. For Type A construct, an initial dormant, non-proliferative period from day 0 to day 3 was followed by a linear growth phase from day 7 to day 21, reaching a plateau after 21 days of culture. On the other hand, the DNA content in Type B gels continues to increase after encapsulation, reaching a maximum value at day 21, with no further changes during the last week of culture. The DNA content per construct increased by 6.8 fold for Type A constructs and 6.5 fold for Type B matrices over 28 days of culture, suggesting an active cell proliferation within the constructs. This proliferative trend reflects the confocal observation after live/dead staining. To gain further insight into the functions of PVFFs cultured in the hydrogel matrices, gene expression related to several ECM proteins, including HYAL2, HAS2, MMP1, ProCOL1 and FN was examined. Freshly isolated PVFFs cultured on TCPS were included as the controls. The results showed that genes related to these ECM proteins were consistently and robustly expressed for cells cultured under all conditions.

Hydroxyproline assay was utilized to quantify the overall collagen content in the cell/gel constructs. In general, Type A constructs contained statistically (p<0.05) higher amount of collagen than Type B constructs at all times except day 3 and day 21. For Type A constructs, the collagen content remained relatively stable until day 21 when an approximately 20% decrease was detected. For Type B constructs, the collagen content gradually decreased over the entire course of culture. Overall, collagen content in both types of constructs at the end of the culture was distinctly (p<0.01) lower than at day 0; a 16% and 20% decrease in collagen content was detected for Type A and Type B constructs, respectively. For Type A matrices incubated under the same conditions in the absence of the encapsulated cells, similar amount of collagen was retained over the entire course of culture. At the end of the culture, the amount of collagen in cell-free hydrogels (1.22 mg/gel) was statistically (p<0.01) higher than that in the cell/gel constructs (0.92 mg/construct). For Type B matrices, hydroxyproline analysis did not reveal any statistical differences between the cell/gel constructs and the cell-free matrices incubated for the same days.

Separately, carbazole assay was applied to monitor the overall HA content in each constructs. For Type A constructs, the total HA content decreased steadily from 0.14 mg at day 0 to 0.02 mg at day 21 and remained essentially unchanged thereafter. For Type B constructs, a rapid decrease (from 1.05 mg at day 0 to 0.45 mg at day 3) was observed during the initial three days of culture and a relatively steady level of HA (0.24-0.28 mg/construct) was maintained from day 14 to day 28. HA content remained significantly higher (p<0.001) in Type B gels than in Type A gels at all times. Control experiments with cell-free hydrogels showed similar profiles of HA retention as a function of culture time for both types of matrices. For Type A gels, the total HA content decreased from 0.23 mg/construct at day 0 to 0.06 mg/construct at day 28. For Type B gels, the total HA content decreased from 1.07 mg at day 0 to 0.34 mg per constructs at day 28. At the end of culture, the overall HA content was slightly higher in cell-free hydrogels than in the corresponding cell/gel constructs for both types of matrices.

The matrix composition was also analyzed by histological staining after 28-days of culture. Histology images of the cell/gel constructs revealed a typical loose connective tissue morphology, containing abundant open, fluid-filled spaces similar to those observed for porcine vocal fold LP. The dense epithelial layer on top of the LP was clearly visible in the porcine tissue. H&E staining showed the presence of scattered PVFFs in the constructs. The seemingly low cellularity observed in the histology samples did not contradict the high cell density suggested by the live/dead assay since the former was an image of a 20-μm thick section whereas the latter was a stacked 3D image. Collagen and HA seemed to be co-localized in both types of constructs. Type A gels were stained brownish yellow by Movat pentachrome staining while Type B gels were stained green due to the relatively higher HA content in Type B gels. Noticeably, both H&E and Movat pentachrome staining revealed that Type B constructs were more diffuse and amorphous in nature than Type A constructs. The presence of elastin in the tissue was confirmed by the dark purple color in the Movat staining. No elastin was detected histologically in the cell/gel constructs.

The viscoelasticity of the composite hydrogels as well as the cell/gel constructs were evaluated using a custom-designed torsional wave apparatus (TWA) at frequencies close to human phonation. All samples were rapidly frozen in liquid nitrogen and were stored and shipped under liquid nitrogen atmosphere prior to the measurements. Good agreement between predicted linear viscoelastic response and experimental data was observed for all samples tested. Two important parameters deduced from the TWA experiments were elastic modulus (G′) and the loss tangent (tan δ: ratio between the loss modulus, G″, and the elastic modulus, G′).

At day 0, the cell/gel constructs and cell-free matrices exhibited similar elastic modulus, for both Type A and Type B gels, respectively. The average G′ and tan (δ) for Type A gels in the absence of PVFFs at a resonance frequency of 65-205 Hz were 1768 Pa and 0.17, respectively. Type B gels, however, were much softer, exhibiting an elastic modulus of 308 Pa and a tan (δ) value of 0.16 at the resonance frequency of 26-60 Hz. The viscoelastic properties of Type A and Type B gels are similar to that of mature and newborn porcine vocal fold LP, respectively. Type A hydrogels without the encapsulated PVFFs exhibited a steady decrease in elastic modulus until day 14, reaching a plateau value of 200 Pa after day 21. On the other hand, Type B gels experienced a dramatic decrease in G′ from day 0 (345 Pa) to day 7 (81 Pa). For each type of matrices, the cell-free hydrogels were significantly (p<0.05) softer than their cell-laden counterparts at all times except day 0.

The evolution of matrix elasticity in the presence of PVFFs as a function of culture time followed distinctly different trends for the types of constructs investigated. Type A constructs were significantly stiffer than Type B constructs at day 0. The stiffness of Type A constructs decreased sharply during the first week of culture, followed by a week of matrix stabilization without dramatic changes in the observed elastic modulus. At the end of the culture, the elastic modulus increased slightly, reaching an average value of 1600 Pa at day 28. Contrarily, the elastic modulus for Type B constructs increased monotonically overtime, varying from an initial value of 345 Pa at day 0 to 970 Pa at day 28. The storage moduli of both constructs appear to be converging to a similar end value. The tan (δ) values remained essentially unchanged over the course of culture for both types of constructs, with or without cells. However, a relatively higher tan (δ) value was observed for the cell-free hydrogels as compared to the same type of constructs with cells and cultured for the same duration.

### Example 5

### First Generation Vocal Fold Bioreactor

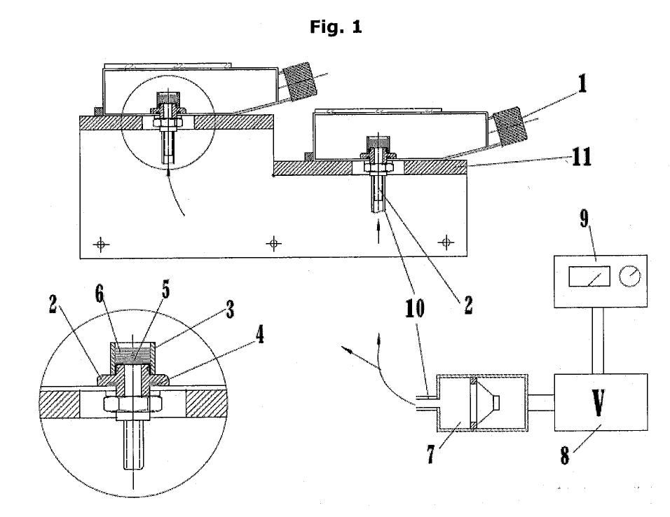

A first generation vocal fold bioreactor (FIG. 1) was constructed to generate well-defined vibrational and shear stresses that closely mimic the mechanical forces exposed to the vocal folds.

In this bioreactor, the vibrational stresses are generated by air forced into motion by a speaker as an acoustic pump 7. The speaker is enclosed in a wooden box having two open holes at the front and back ends. The speaker is driven by a function generator 9 (Agilent 33220A, Agilent Technologies) through a power amplifier 8 (Pyle pro PT-2400, Brooklyn, N.Y.). Two parallel replaceable vibration flasks 1 are anchored to an acrylic stage 11. Each flask has an open hole on the bottom. The acrylic stage 11 is installed with two connecting tubes 2 via washers 4. The surface of the connecting tube 2 is covered with cells encapsulated within the hydrogel matrix and anchored on the collagen I-coated silicone (PDMS) membrane 5 (SFM-CI, Flexcell, Hillsborough, N.C.). A conducting tube 10 connects one open hole on the wooden box with the open hole on the bottom of a flask. A rigid plastic anchoring tube 3 (Tygon S-50-HL, Saint-Gobain, Akron, Ohio) effectively secures and seals the membrane on the connecting tube 2 at the open hole of the flask 1, creating a small vibration chamber where cell culture media 6 can be added into the flask 1.

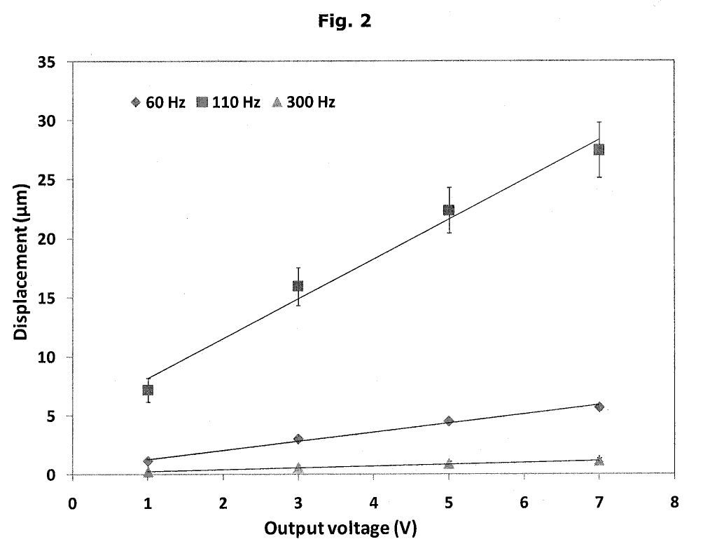

The biomechanical stimulations generated include: normal vibration, in-plane strain and fluid shear, closely mimicking a variety of mechanical forces encountered by vocal fold lamina propria. The membrane vibrates up and down as a result of air pulsed by the speaker. The normal displacement was assessed using a Laser Doppler Vibrometer (Polytec). The laser was positioned perpendicularly to the membrane at a predetermined location. The reflected beam was detected by the monitor and the vibrational velocity was converted to normal displacement and frequency using Fourier Transform. The membrane displacement was controlled through the voltage, and the frequency selected on the function generator. As the output voltage increases, the membrane displacement increases linearly (FIG. 2). When the output voltage is kept constant, the membrane displacement decreases as the frequency increases. Thus, the amplitude of the vibrations is controlled by varying the output voltage. To maintain the same displacement, a higher output voltage is required for vibrations at higher frequencies.

### Example 6

### Second Generation Vocal Fold Bioreactor

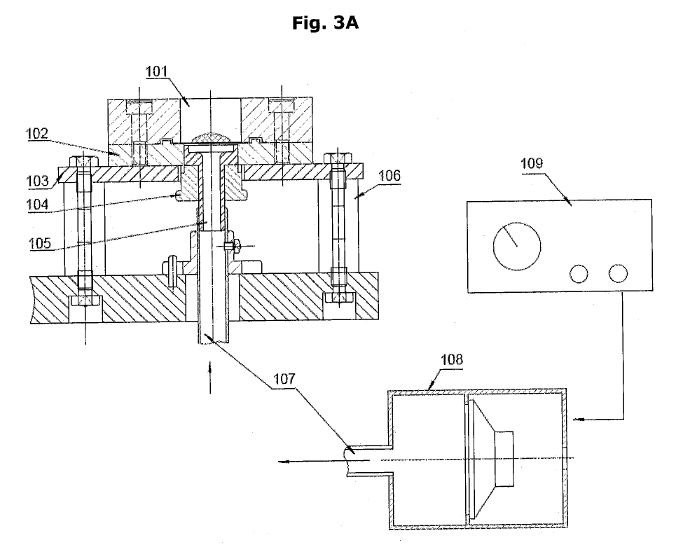

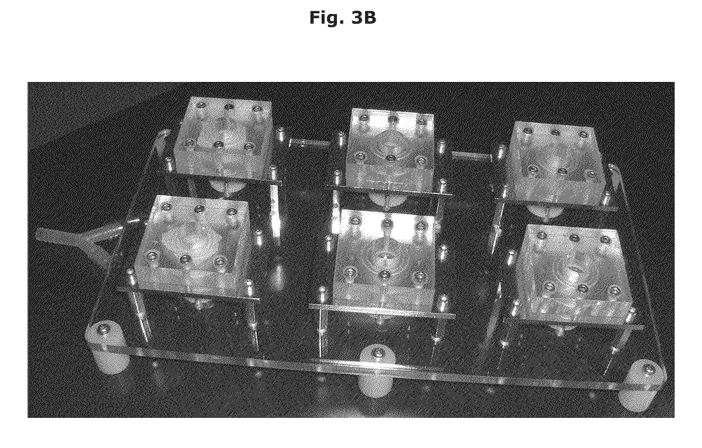

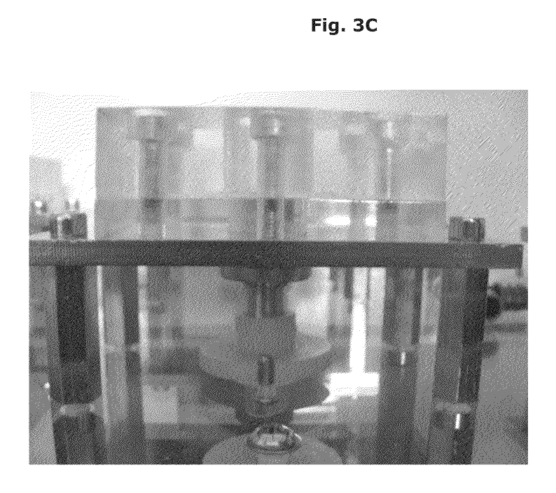

A second generation vocal fold bioreactor (FIGS. 3A-3C) was constructed to overcome some limitations in the first generation bioreactor as described in Example 5. In particular, the second generation bioreactor is capable of adjusting the degree of pre-stress (strain) introduced to the membrane and providing easy capture of cell images without disrupting the cells.

In this bioreactor, the vibrational stress is generated by air forced into motion via an acoustic pump in a casing. The vibration is translated into the chamber using a flexible plastic tubing. The overall bioreactor design includes a PDMS membrane sandwiched between a pair of mountable acrylic blocks (top acrylic block enclosing a vibration chamber 101 and bottom acrylic block 102) containing matching ridges and grooves that lock the membrane in place. Four corner screws connect the top and the bottom plates, essentially creating a water-tight vibration chamber (24 mm in diameter and 18 mm in height) that allows for cells to be cultured. The opening of the chamber is covered with a clear adhesive tape that effectively seals off the flask during culture. The pair of mountable acrylic blocks are anchored on a metal platform 103 made of nickel-coated brass using the long center screws. Upon completion of cell culture, the acrylic blocks containing cultured cells and media are readily removed from the platform by loosening these screws, allowing cells to be directly imaged without having to disassemble the acrylic plates and disrupting the cultured cells.

The metal frame contains a hole in the middle that can accommodate a rotating knob 104 with a 10-mm matching opening in the middle. A T-shaped metal tube 105 sits freely through the rotating knob or adjustable knob 104. Each revolution the knob 104 makes creates a height increase for the T-shaped metal connecting tube 105, pushing the membrane upwards. The in-plane strain generated on the membrane is therefore proportional to the relative height of the T-shaped tube 105. Each tube is held in place and sealed tightly at its entrance by nylon bolts and joints. The end of T-shaped tube 105 is connected to the acoustic pump casing via a plastic conducting tube 107 through the main frame. The main frame containing six parallel assemblies fits easily in a standard CO₂ incubator. To prevent the plastic conducting tube 107 from being bent after it goes through the main acrylic frame, glass tube with a 90 degree turn is used to connect the tubing coming out of the T-shaped metal tube and the tube connected to the acoustic pump 108. The vibration amplitude and frequency are controlled by a power amplifier coupled to a function generator 109 that is capable of scanning a large frequency range (20-200 KHz). The entire board fits easily into an incubator, and the electronic components remain outside of the incubator. The height of the T-shaped metal connecting tube 105, relative to the surface of the metal frame, is a critical parameter for controlling the pre-stress on the membrane. The supporting column 106 provides structural support and creates space for incorporating various tubings underneath the acrylic vibration chamber.

### Example 7

### Neonatal Foreskin Fibroblasts and Human Mesenchymal Stem Cells in 2D Dynamic Culture

Studies were carried out to evaluate cellular responses to high frequency vibrational stimulations. In particular, neonatal foreskin fibroblasts (NFFs, ATCC) and mesenchymal stem cells (MSCs, Lonza) were cultured in the first generation or the second generation vocal fold bioreactors, respectively. NFFs and MSCs were maintained in fibroblast basal media and mesenchymal stem cell growth media, supplemented with respective kits provided by the venders. Cells were maintained at 37° C. with 5% CO₂ and were used at passage 4 or 5. The bioreactors were constructed and characterized as described in Example 5 and 6. Cells were seeded directly on the collagen-coated PDMS membrane, and the medium was added to the culture chamber. The culture chambers and the stages were placed in a cell culture incubator and the vibrational tube was connected to the acoustic pump (located outside the incubator) via a sterile filter through a pre-established hole on the incubator wall.

All components of the bioreactors were sterilized under germicidal UV lamp for 6 h before being assembled for the dynamic culture. Cells were seeded on collagen I-coated silicone membranes at a density of 50,000 cells/cm². Cells were pre-cultured in the bioreactor under static conditions for 24 h before vibrational stimulations were initiated. NFFs were subjected to 1 h vibration at frequencies of 60 Hz, 110 Hz, and 300 Hz with varying an output voltage varying from 1 V to 7 V. The stimulated cells were allowed to rest for 6 h before being harvested for analysis. Separately, MSCs were subjected to 1 h-on-1 h-off for a total of 12 hr at an applied frequency of 120 Hz and output voltage of 3V. An output voltage of 3 V corresponds to a normal displacement of 100 μm at the center of the membrane for the second generation bioreactor.

After the dynamic culture was terminated, cell morphology was analyzed by F-actin staining. Cells were rinsed two times with Dubellco's Phosphate Buffer Saline (DPBS, Gibco) before being fixed with 4% paraformaldehyde solution for 20 min (in DPBS, Electron Microscopy Sciences). Cells were then washed two times with a washing buffer (0.05% Tween 20 in DPBS, Fisher), permeabilized for 5 min with 0.1% Triton X-100 (in DPBS, Fisher), rinsed again two times with the washing buffer, blocked for 30 min with 1% Bovine Serum Albumin (in DPBS, Jackson Immunoresearch), washed one last time with washing buffer, and stained overnight with Alexa Fluor 488 Phalloidin (1:1000, Invitrogen), and DRAQ5™ (1:2000, Alexis Biochemicals). On the next day, membranes were rinsed three times with DPBS, before being carefully removed from the bioreactor and mounted on microscope slides with Gel Mount (Biomeda/Electron Microscopy Sciences). Imaging was performed using an LSM510 Axiovert confocal microscope (Zeiss). No noticeable changes in cell cytoskeleton organization due to vibration at different frequencies were observed, and no apparent cell damage was observed.

Cell Titer Blue (Promega) was used to quantify the total number of cells cultured under static and dynamic conditions. Before vibration, 100 μl of cell titer blue was added to the bioreactor wells. Upon completion of dynamic culture, 100 μl of the sample was transferred to a 96-well plate and the fluorescence (excitation: 550 nm and emission: 590 nm) was recorded using a microplate reader (Perkin Elmer). The cell density under dynamic culture conditions was normalized to that of static controls. A relatively stable normalized cell number was found before and after vibration. The vibrations, at any chosen frequency, did not seem to cause the cells to be lifted off or undergo apoptosis. Accordingly, the results shown by qPCR and F-actin were representative of the entire cell population which was originally seeded onto the membrane.

Quantitative polymerase chain reaction (qPCR) was carried out to analyze mRNA expression of ECM-related genes. Upon completion of the dynamic culture experiments, cells were collected and stored at −80° C. for further analysis. The RNA was extracted from the cells using a Qiagen RNeasy kit according to the manufacturer's instructions, and the RNA concentration was determined using a ND-1000 Spectrophotometer (Nanodrop). Two micrograms of RNA from each sample was purified with gDNA elimination and reverse transcripted into cDNA using QuantiTect Rev. Transcription kit (Qiagen). The mRNA levels for type I collagen (Coll 1), fibronectin (FN), matrix metallopeptidase 1 (MMP1), cyclooxygenase 2 (COX2), CD44, tissue inhibitor of metallopeptidase 1 (TIMP1), elastin, HA synthase 1 (HAS1) and glyceraldehydes-3-phosphate dehydrogenase (GAPDH) were analyzed by quantitative polymerase chain reaction (qPCR) using an Applied Biosystems 7000 real-time PCR system with 25 μl of SYBR green reagents (Applied Biosystems). GAPDH was used as the housekeeping gene and the relative mRNA expression was calculated with respect to GAPDH and the static controls, using the ΔΔCt method, where fold difference was calculated using the expression 2^{(-ΔΔCt)} (primer efficiencies were verified prior to using them, and were all around 2, allowing the use of the above 2^{(-ΔΔCt)} expression instead of the more formal Pfaffl equation). Primer sequences were chosen on the basis of sequence information in Genbank Database. The validity of the primers was verified by running a traditional PCR on the sample cDNA. The PCR amplification was performed for 35 cycles using GoTaq Green master mix (Promega), and a MyGene Series Peltier thermal cycler (MG96G, Long Gene). The cycling conditions were as follows: initial denaturation at 94° C. for 3 min, denaturation at 94° C. for 30 sec, annealing at 55° C. for 30 sec, extension at 72° C. for 1 min, and a final extension at 72° C. for 10 min. PCR products were electrophoresed on 1.2% agarose gels with ethidium bromide, and the images were captured with an Alpha Imager (Alpha Innotech Corporation). All PCR products were detected at the predicted locations.

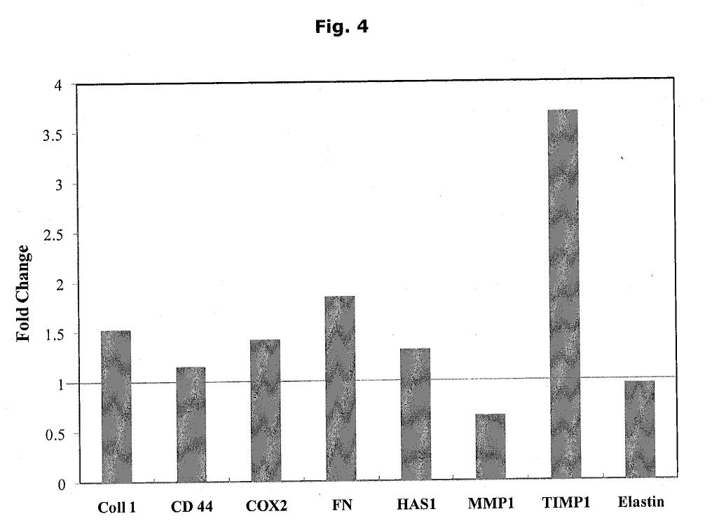

While moderate changes in the mRNA levels of ECM-related genes were observed for NFFs subjected to 1 h vibration using the first generation bioreactor, significant up-regulation (relative to the static control) of Coll 1, FN, HAS1 and TIMP1 were observed for MSCs subjected to alternating on and off vibrations generated with the second generation bioreactor (FIG. 4). Moreover, MSCs cultured under dynamic conditions expressed lower amount of MMP1 relative to those cultured under static conditions. Finally, the elastin expression level remains unchanged. The results suggest that the cellular responses to vibrational stimulations are strongly dependant on (1) the cell types; (2) the vibration pattern and duration and (3) the frequency and amplitude of vibration. MSCs cultured under dynamic conditions exhibited enhanced potential for ECM production. The results confirm that the vibrational stimulation close to human phonation is a potent regulator of gene expression and cell functions.

### Example 8

### Mesenchymal Stem Cells in 3D Dynamic Culture

This is a prophetic example. Experiments have been designed to synthesize vocal fold specific artificial ECM that recaptures the characteristics of their natural analog by incorporation of the following features: (1) in situ formation of 3D hydrogel scaffolds by simple mixing; (2) in situ encapsulation of biological molecules and cells; (3) presentation of topological features within the scaffold due to the presence of two levels of crosslinking; (4) modulation of the elastic characteristics of the hydrogels by design, (5) local presentation of bound adhesion factors and controlled release of growth factor morphogens, and (6) remodeling of the gel matrix by the local enzymatic processes associated with cell infiltration. The second generation bioreactor as described in Example 7 will be used for generating physiologically relevant mechanical stimulations, with the vibration frequency, normal displacement and dynamic in-plane strain readily adjustable. The vibrational stress will be generated by the air driven into motion by an acoustic pump, closely mimicking the natural tissue where the sound is produced by wave-like motion of the lamina propria forced into vibration by the air from the lung. 3D culturing of MSCs in the HA-based artificial ECM in the presence of external mechanical stimulation will be evaluated. Cells cultured on 2D PDMS surface in the absence of the mechanical stimulation will be used as the control.

The biomimetic environment exhibits distinct features: insoluble, 3D matrix, soluble factors, and the external mechanical signals. The interplay of these factors will likely result in synergistic regulatory effect on MSC differentiation and tissue assembly. Five experiments will be set up: (1) monolayer culture (TCPS), (2) monolayer culture+growth factor (GF) in the media, (3) 3D culture in Type B HA/collagen gel, (4) 3D culture in HA DXNs without GF, and (5) 3D culture in HA DXNs with immobilized GF. Each experiment will carried out in a static culture for evaluating matrix effect as well as a dynamic culture to evaluate combination effect of matrix effect and mechanical effect.

2D monolayer culture will be conducted in order to determine the relevance of FGF-2 and HGF in modulate MSC behaviors in terms of the desired dosage and doping intervals since the HA-based matrix provides additional topographical cues that may be conducive to MSC differentiation.

The static culture will be carried out using the second generation vocal fold bioreactor in the absence of vibration in order to obtain proper controls for later comparison.

The effect of dynamic culture will be evaluated on MSC differentiation using a 2D monolayer culture format in the presence of mechanical signals generated using the second generation vocal fold bioreactor. After trypsinization and cell plating, MSCs will be allowed to grow under static conditions for one day prior to the introduction of mechanical stimulations. In initial experiments, MSCs will be subjected to 1 h-on 1 h-off vibrations for 6, 12 and 24 h in order to obtain information regarding the short term effects of mechanical vibration. The maximum normal displacement (at the center of the membrane) will be maintained in the range of 0.1-1 mm, and the in-plane, dynamic strain will be varied from 10% to 30%. This can be achieved by adjusting the output voltage of the power amplifier and/or pre-strain on the membrane prior to the on set of vibration. In the experiment, the applied vibration frequency will be in the range of 100-400 Hz, while the max variation of shear strain will be maintained to be 30%, closely mimicking the mechanical environment in natural vocal fold.

Growth factors will be supplemented into the culture media at dosage levels at 20-100 ng/ml. Upon completion of the dynamic culture, the acrylic blocks containing the sandwich membrane and the attached cells will be removed from the stage for confocal imaging after immunostaining. The cell morphology, cytoskeleton organization and focal adhesion will be closely monitored. Cell proliferation will be analyzed by cell titer blue and WST assays. The fibroblastic phenotype will be quantified by gene array analysis. Western analysis will be used to quantify the protein production. Results will be compared to MSCs cultured under the same conditions without the vibrational stimulations.

Next, MSCs will be encapsulated in the hydrogel at a seeding density of 10⁵ cells/ml. The mixture will be loaded into the vibration chamber and be allowed to gel for 30 min before the media is added to the chamber. The construct will be cultured under static conditions for up to 7 days prior to the initial mechanical stimulation. The regimens of the stimulation will be systematically varied to include those outlined in Table 1, which set forth the experimental conditions for 3D dynamic culture. Cell triggered tension has been shown to deform the latent growth factor complex, causing the sequestered growth factors to be released to the surrounding milieu. The HGPs containing the latent GF/PlnDI complexes are embedded in a secondary matrix. The coupling between the HGPs and the secondary network allows for the mechanical forces be transmitted to the particles and potentially dissociate the GF/PlnDI complex, resulting in a transient high concentration of growth factors in close proximity of the encapsulated MSCs. It is expected that, MSCs exposed to such concerted action of soluble factors and biomechanical signals will not only undergo directed differentiation but also maintain their “synthetic” phenotype. Furthermore, the mechanical stimulations provided by the bioreactor will not only foster nutrient and waste diffusion, but also stimulate the production of native LP ECM.

TABLE 1
Experimental Conditions For 3D Dynamic Culture

| Condition | Parameter |
|---|---|
| Vibration frequency (Hz) | 0, 125, 210, 350 |
| Max normal displacement (mm) | 0.2-2 |
| Shear strain (in plane) (%) | 10-30% |
| Stimulation pattern | 10%-on, 90%-off or |
| | 50%-on 50% off |
| Total stimulation time per day (h) | 2-4 |
| Days of vibration | 14 |
| Days of Static culture prior to vibration | 0-7 |

Throughout the investigations, the immortalized vocal fold fibroblast cell line will be cultured under the same conditions and be used as controls. Upon completion of the experiments, cell viability and proliferation will be systematically assessed using standard cell proliferation assay kit. Cytoskeletal changes due to prolonged vibration will be examined. For this purpose, actin organization will be recorded using a Nikon Optiphot II microscope through 100× objective after the fibroblasts are fixed in paraformaldehyde, permeablized with Triton-X100, and stained with FITC-phalloidin. Mechanical loading increases ECM protein production, modulates ECM turnover, and regulates ECM homeostasis in various tissues. Therefore, ECM protein production will be qualitatively analyzed using immunofluorescence and quantified by Western analysis or ELISA. These observations will be compared to static cells that have been placed under the same environmental conditions.

The vocal fold tissue construct will be subjected to biological and histological analysis. The engineered vocal folds will be subjected to traditional dynamic mechanical measurements as well as torsional wave experiments in order to evaluate the evolution of their mechanical properties over time. Parallel histological assessment will be carried out in order to understand the structural-function relationship for the engineered vocal fold tissues. Promising samples will be tested using the torsional wave apparatus, Viscoelastic properties of human LP tissues collected will be used as the benchmarks for comparison.

Various terms relating to the systems, methods, and other aspects of the present invention are used throughout the specification and claims. Such terms are to be given their ordinary meaning in the art unless otherwise indicated.

The present invention is not limited to the embodiments described and exemplified above, but is capable of variation and modification within the scope and range of equivalents of the appended claims.

What is claimed:

1. A method for inducing in vitro differentiation of stem cells into vocal fold fibroblast-like cells comprising (a) culturing stem cells encapsulated in a matrix, wherein the matrix comprises hyaluronic acid (HA)-based hydrogel particles (HGPs) covalently cross-linked by a water soluble polymer, and one or more growth factors bound to the HA-based HGPs, and (b) delivering a high frequency vibration with in-plane shear stress to the stem cells, wherein the matrix releases the one or more growth factors in a controlled manner effective to induce differentiation of the stem cells into vocal fold fibroblast-like cells in the presence of the vibration.

2. The method of claim 1, wherein the stem cells are mesenchymal stem cells (MSCs).

3. The method of claim 1, wherein the matrix further comprises collagen.

4. The method of claim 1, wherein the matrix further comprises a biologically active compound selected from the group consisting of an antifibrotic drug, a cell adhesive peptide, and morphogenic factors.

5. A biomaterial produced by the method of claim 1.

6. The biomaterial of claim 5, comprising the matrix, the stem cells, the vocal fold fibroblast-like cells, or a combination thereof.

7. The vocal fold fibroblast-like cells produced by the method of claim 1.

8. A method for regenerating a vocal fold tissue in an animal comprising transferring the vocal fold fibroblast-like cells of claim 7 into vocal fold of the animal.

9. A method for inducing in vitro generation of a vocal fold-like tissue from cultured cells comprising (a) culturing cells encapsulated in a matrix, wherein the matrix comprises hyaluronic acid (HA)-based hydrogel particles (HGPs) covalently cross-linked by a water soluble polymer, and one or more growth factors bound to the HA-based HGPs, and (b) delivering a high frequency vibration with in-plane shear stress to the cells, wherein the matrix releases the one or more growth factors in a controlled manner effective to induce generation of a vocal fold-like tissue from the cultured cells in the presence of the vibration.

10. The method of claim 9, wherein the cells are selected from the group consisting of primary vocal fold fibroblast cells, skin fibroblast cells and mesenchymal stem cells.

11. The vocal fold-like tissue produced by the method of claim 9.

12. A method for regenerating vocal fold in an animal comprising transferring the vocal fold-like tissue of claim 11 into vocal fold of the animal.

13. A matrix comprising (a) hyaluronic acid (HA)-based hydrogel particles (HGPs) covalently cross-linked by a water soluble polymer, and (b) one or more growth factors bound to the HA-based HGPs, wherein the matrix is capable of releasing the one or more growth factors in a controlled manner.

14. The matrix of claim 13, wherein the water soluble polymer is selected from the group consisting of a hyaluronic acid (HA) derivative carrying a hydrazide (HAADH) group, a HA derivative carrying an aldehyde (HAALD), a HA modified with glycidyl methacrylate (GMA), and a HA modified with thiol.

15. The matrix of claim 13, further comprising stem cells encapsulated in the matrix, wherein the controlled manner is effective to induce differentiation of the stem cells into specialized cells.

16. The matrix of claim 13, further comprising cultured cells encapsulated in the matrix, wherein the controlled manner is effective to induce generation of a tissue from the cultured cells.

17. A method, comprising culturing cells in the matrix of claim 13.

18. A device for generating and delivering a high frequency vibration with in-plane shear stress to cultured cells comprising (a) a chamber for culturing cells on a membrane, (b) an acoustic pump for generating the high frequency vibration by forcing air into motion, and (c) a tube connecting the acoustic pump with the membrane for delivering the high frequency vibration with in-plane shear stress to the cultured cells via the membrane.

19. The device of claim 18, wherein the high frequency is in the range from about 15 to about 20,000 Hz.

20. A method, comprising culturing cells in the device of claim 18, wherein the cultured cells are vibration-sensitive cells.

21. The method of claim 20, further comprising generating a vibration-sensitive tissue from the cultured cells.

22. An apparatus comprising (a) a matrix comprising hyaluronic acid (HA)-based hydrogel particles (HGPs) covalently cross-linked by a water soluble polymer, and one or more growth factors bound to the HA-based HGPs, wherein the matrix is capable of releasing the one or more growth factors in a controlled manner, and (b) a device for generating and delivering a high frequency vibration with in-plane shear stress to the matrix.

23. The apparatus of claim 22, wherein the device comprises (a) a cell culture chamber having the matrix on a membrane, (b) an acoustic pump for forcing air into motion to generate the high frequency vibration, and (c) a tube connecting the acoustic pump with the membrane for delivering the high frequency vibration with in-plane shear stress to the matrix via the membrane.

24. The apparatus of claim 22, further comprising stem cells encapsulated in the matrix and subject to the high frequency vibration with in-plane shear stress, wherein the controlled manner is effective to induce differentiation of the stem cells into vibration-sensitive specialized cells.

25. The apparatus of claim 22, further comprising cultured cells encapsulated in the matrix and subject to the high frequency vibration with in-plane shear stress, wherein the controlled manner is effective for generating a vibration-sensitive tissue from the cultured cells.
